SEQUENCE LISTING (2 sequences)

SEQ ID NO: 1 - protein, 281 aa, Homo sapiens [sequence omitted]

SEQ ID NO: 2 - DNA, 1042 bp, Homo sapiens
  CDS (91)..(933)
  modified_base (447)..(447): a, c, t or g
tttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtccncagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaactgacctggaaagaaaaagcaataacctcaaagtgactattcagttttcaggatgatacactatgaagatgtttcaaaaaatctgaccaaaacaaacaaacagaaa

(19) United States
(12) Patent Application Publication
(10) Pub. No.: US 2017/0290883 A1
(43) Pub. Date: Oct. 12, 2017

(54) VASCULAR DISRUPTION AGENTS AND USES THEREOF

(71) Applicant: Genentech, Inc., South San Francisco, CA (US)

(72) Inventor: Avi J. Ashkenazi, San Mateo, CA (US)

(73) Assignee: Genentech, Inc., South San Francisco, CA (US)

(21) Appl. No.: 15/356,015

(22) Filed: Nov. 18, 2016

Related U.S. Application Data
(63) Continuation of application No. 14/115,186, filed on Feb. 26, 2014, now abandoned.
(60) Provisional application No. 61/482,035, filed on May 3, 2011.

(51) Int. Cl. A61K 38/16; A61K 39/00; A61K 38/02

(52) U.S. Cl. CPC A61K 38/16; A61K 38/02; A61K 39/0005

(57) ABSTRACT

Uses of Apo2L/TRAIL polypeptides and death receptor agonist antibodies to disrupt tumor associated vasculature are provided. Methods of treating cancer in mammals, kits, and articles of manufacture are also provided.

### CROSS-REFERENCE TO RELATED APPLICATIONS

This continuing application is a Continuation of U.S. application Ser. No. 14/115,186 filed on Nov. 1, 2013, which is a National Stage application of PCT/US2012/036181 filed on May 2, 2012, which claims the benefit of U.S. provisional patent application No. 61/482,035 filed on May 3, 2011, each of which is incorporated by reference in its entirety.

### FIELD OF THE INVENTION

The present invention relates to proapoptotic receptor agonists (PARAs) and uses of such PARAs to disrupt tumor associated vasculature. In particular, the invention relates to Apo2L/TRAIL compositions and uses of such Apo2L/TRAIL compositions to disrupt vasculature in mammalian cells or tissue, particularly in mammalian tumor-associated vasculature. The invention also relates to methods of disrupting vasculature in mammals and to methods of treating disorders such as cancer in mammals. Kits and articles of manufacture are also included.

### BACKGROUND OF THE INVENTION

Various ligands and receptors belonging to the tumor necrosis factor (TNF) superfamily have been identified in the art. Included among such ligands are tumor necrosis factor-alpha (“TNF-alpha”), tumor necrosis factor-beta (“TNF-beta” or “lymphotoxin-alpha”), lymphotoxin-beta (“LT-beta”), CD30 ligand, CD27 ligand, CD40 ligand, OX-40 ligand, 4-1BB ligand, LIGHT, Apo-1 ligand (also referred to as Fas ligand or CD95 ligand), Apo-2 ligand (also referred to as Apo2L or TRAIL), Apo-3 ligand (also referred to as TWEAK), APRIL, OPG ligand (also referred to as RANK ligand, ODF, or TRANCE), and TALL-1 (also referred to as BlyS, BAFF or THANK) (See, e.g., Ashkenazi, Nature Review, 2:420-430 (2002); Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997) Wallach, Cytokine Reference, Academic Press, 2000, pages 377-411; Locksley et al., Cell, 104:487-501 (2001).

Induction of various cellular responses mediated by such TNF family ligands is typically initiated by their binding to specific cell receptors. Some, but not all, TNF family ligands bind to, and induce various biological activity through, cell surface “death receptors” to activate caspases, or enzymes that carry out the cell death or apoptosis pathway (Salvesen et al., Cell, 91:443-446 (1997). Included among the members of the TNF receptor superfamily identified to date are TNFR1, TNFR2, TACI, GITR, CD27, OX-40, CD30, CD40, HVEM, Fas (also referred to as Apo-1 or CD95), DR4 (also referred to as TRAIL-R1), DR5 (also referred to as Apo-2 or TRAIL-R2), DcR1, DcR2, osteoprotegerin (OPG), RANK and Apo-3 (also referred to as DR3 or TRAMP) (see, e.g., Ashkenazi, Nature Reviews, 2:420-430 (2002); Ashkenazi and Dixit, Science, 281:1305-1308 (1998); Ashkenazi and Dixit, Curr. Opin. Cell Biol., 11:255-260 (2000); Golstein, Curr. Biol., 7:750-753 (1997); Wallach, Cytokine Reference, Academic Press, 2000, pages 377-411; Locksley et al., Cell, 104:487-501 (2001)).

Most of these TNF receptor family members share the typical structure of cell surface receptors including extracellular, transmembrane and intracellular regions, while others are found naturally as soluble proteins lacking a transmembrane and intracellular domain. The extracellular portion of typical TNFRs contains a repetitive amino acid sequence pattern of multiple cysteine-rich domains (CRDs), starting from the NH₂-terminus.

The ligand referred to as Apo-2L or TRAIL was previously identified as a member of the TNF family of cytokines. (see, e.g., Wiley et al., Immunity, 3:673-682 (1995); Pitti et al., J. Biol. Chem., 271:12697-12690 (1996); WO 97/01633; WO 97/25428; U.S. Pat. No. 5,763,223 issued Jun. 9, 1998; U.S. Pat. No. 6,284,236 issued Sep. 4, 2001). The full-length native sequence human Apo2L/TRAIL polypeptide is a 281 amino acid long, Type II transmembrane protein. Some cells can produce a natural soluble form of the polypeptide, through enzymatic cleavage of the polypeptide's extracellular region (Mariani et al., J. Cell. Biol., 137:221-229 (1997)). Crystallographic studies of soluble forms of Apo2L/TRAIL reveal a homotrimeric structure similar to the structures of TNF and other related proteins (Hymowitz et al., Molec. Cell, 4:563-571 (1999); Cha et al., Immunity, 11:253-261 (1999); Mongkolsapaya et al., Nature Structural Biology, 6:1048 (1999); Hymowitz et al., Biochemistry, 39:633-644 (2000)). Apo2L/TRAIL, unlike other TNF family members however, was found to have a unique structural feature in that three cysteine residues (at position 230 of each subunit in the homotrimer) together coordinate a zinc atom, and that the zinc binding is important for trimer stability and biological activity. (Hymowitz et al., supra; Bodmer et al., J. Biol. Chem., 275:20632-20637 (2000)).

Soluble forms of Apo2L/TRAIL have also been reported to induce apoptosis in a variety of cancer cells, including colon, lung, breast, prostate, bladder, kidney, ovarian and brain tumors, as well as melanoma, leukemia, and multiple myeloma (see, e.g., Wiley et al., supra; Pitti et al., supra; U.S. Pat. No. 6,030,945 issued Feb. 29, 2000; U.S. Pat. No. 6,746,668 issued Jun. 8, 2004; Rieger et al., FEBS Letters, 427:124-128 (1998); Ashkenazi et al., J. Clin. Invest., 104:155-162 (1999); Walczak et al., Nature Med., 5:157-163 (1999); Keane et al., Cancer Research, 59:734-741 (1999); Mizutani et al., Clin. Cancer Res., 5:2605-2612 (1999); Gazitt, Leukemia, 13:1817-1824 (1999); Yu et al., Cancer Res., 60:2384-2389 (2000); Chinnaiyan et al., Proc. Natl. Acad. Sci., 97:1754-1759 (2000)). In vivo studies in murine tumor models further suggest that Apo2L/TRAIL, alone or in combination with chemotherapy or radiation therapy, can exert substantial anti-tumor effects (see, e.g., Ashkenazi et al., supra; Walzcak et al., supra; Gliniak et al., Cancer Res., 59:6153-6158 (1999); Chinnaiyan et al., supra; Roth et al., Biochem. Biophys. Res. Comm., 265:1999 (1999); PCT Application US/00/15512; PCT Application US/01/23691). In contrast to many types of cancer cells, most normal human cell types appear to be resistant to apoptosis induction by certain recombinant forms of Apo2L/TRAIL (Ashkenazi et al., supra; Walzcak et al., supra). Jo et al. has reported that a polyhistidine-tagged soluble form of Apo2L/TRAIL induced apoptosis in vitro in normal isolated human, but not non-human, hepatocytes (Jo et al., Nature Med., 6:564-567 (2000); see also, Nagata, Nature Med., 6:502-503 (2000)). Li et al. has reported that a recombinant preparation of human TRAIL triggered apoptosis in cultured human endothelial cells (Li et al., J. Immunol., 171:1526-1533 (2003)). It is believed that certain recombinant Apo2L/TRAIL preparations may vary in terms of biochemical properties and biological activities on diseased versus normal cells, depending, for example, on the presence or absence of a tag molecule, zinc content, and % trimer content (See, Lawrence et al., Nature Med., Letter to the Editor, 7:383-385 (2001); Qin et al., Nature Med., Letter to the Editor, 7:385-386 (2001)).

Apo2L/TRAIL has been found to bind at least five different receptors. At least two of the receptors which bind Apo2L/TRAIL contain a functional, cytoplasmic death domain. One such receptor has been referred to as “DR4” (and alternatively as TR4 or TRAIL-R1) (Pan et al., Science, 276:111-113 (1997); see also WO98/32856 published Jul. 30, 1998; WO99/37684 published Jul. 29, 1999; WO 00/73349 published Dec. 7, 2000; U.S. Pat. No. 6,433,147 issued Aug. 13, 2002; U.S. Pat. No. 6,461,823 issued Oct. 8, 2002, and U.S. Pat. No. 6,342,383 issued Jan. 29, 2002).

Another such receptor for Apo2L/TRAIL has been referred to as DR5 (it has also been alternatively referred to as Apo-2; TRAIL-R or TRAIL-R2, TR6, Tango-63, hAPO8, TRICK2 or KILLER) (see, e.g., Sheridan et al., Science, 277:818-821 (1997); Pan et al., Science, 277:815-818 (1997); WO98/51793 published Nov. 19, 1998; WO98/41629 published Sep. 24, 1998; Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999; US 2002/0072091 published Aug. 13, 2002; US 2002/0098550 published Dec. 7, 2001; U.S. Pat. No. 6,313,269 issued Dec. 6, 2001; US 2001/0010924 published Aug. 2, 2001; US 2003/01255540 published Jul. 3, 2003; US 2002/0160446 published Oct. 31, 2002; US 2002/0048785 published Apr. 25, 2002; U.S. Pat. No. 6,342,369 issued February, 2002; U.S. Pat. No. 6,569,642 issued May 27, 2003; U.S. Pat. No. 6,072,047 issued Jun. 6, 2000; U.S. Pat. No. 6,642,358 issued Nov. 4, 2003; U.S. Pat. No. 6,743,625 issued Jun. 1, 2004). Like DR4, DR5 is reported to contain a cytoplasmic death domain and be capable of signaling apoptosis upon ligand binding (or upon binding a molecule, such as an agonist antibody, which mimics the activity of the ligand). The crystal structure of the complex formed between Apo-2L/TRAIL and DR5 is described in Hymowitz et al., Molecular Cell, 4:563-571 (1999).

Upon ligand binding, both DR4 and DR5 can trigger apoptosis independently by recruiting and activating the apoptosis initiator, caspase-8, through the death-domain-containing adaptor molecule referred to as FADD/Mortl [Kischkel et al., Immunity, 12:611-620 (2000); Sprick et al., Immunity, 12:599-609 (2000); Bodmer et al., Nature Cell Biol., 2:241-243 (2000)].

Apo2L/TRAIL has been reported to also bind those receptors referred to as DcR1, DcR2 and OPG, which believed to function as inhibitors, rather than transducers of signaling (see, e.g., DCR1 (also referred to as TRID, LIT or TRAIL-R3) [Pan et al., Science, 276:111-113 (1997); Sheridan et al., Science, 277:818-821 (1997); McFarlane et al., J. Biol. Chem., 272:25417-25420 (1997); Schneider et al., FEBS Letters, 416:329-334 (1997); Degli-Esposti et al., J. Exp. Med., 186:1165-1170 (1997); and Mongkolsapaya et al., J. Immunol., 160:3-6 (1998); DCR2 (also called TRUNDD or TRAIL-R4) [Marsters et al., Curr. Biol., 7:1003-1006 (1997); Pan et al., FEBS Letters, 424:41-45 (1998); Degli-Esposti et al., Immunity, 7:813-820 (1997)], and OPG [Simonet et al., supra]. In contrast to DR4 and DRS, the DcR1 and DcR2 receptors do not signal apoptosis.

Although certain cancer cells undergo apoptosis in response to death receptor activation, many exhibit partial or total resistance Yang et al., Curr. Opin. Cell Biol., (2010). Most preclinical studies with proapoptotic receptor agonists (“PARAs”) have relied on cultured human cancer cells or xenografted human tumors grown in mice. However, less is known about the effects of activating proapoptotic receptor pathways in spontaneous or syngeneic tumors. In particular, effects on the tumor microenvironment in animal models have not been well understood, as most PARAs target human death receptors but not the mouse counterparts (Ashkenazi et al., Nat. Rev. Drug Disc., 7:1001-1012 (2008)). Previous studies have used MD5.1, an antibody directed against murine DR5 (or TRAIL-R), the only Apo2L/TRAIL death receptor present in the mouse. MD5.1 is reported to induce apoptosis of cancer cells in vitro, but its tumoricidal efficacy in vivo may be contingent on aspects of innate and adaptive immunity (Takeda et al., J. Exp. Med., 199:437-448 (2004); Uno et al., Nat. Med., 12:693-698 (2006); Frew et al., Proc. Natl. Acad. Sci., 105:11317-11322 (2008); Haynes et al., J. Immunol., 185:532-541 (2010)).

### SUMMARY OF THE INVENTION

A functioning vascular network is critical for the growth and survival of tumors and cancerous cells, and therapeutic agents that target the characteristics of tumor blood vessels represent a novel approach to anticancer therapy. The present disclosure provides for and describes a novel role for death receptor 5 (“DR5”) signaling in the tumor-associated endothelial cell compartment in mammals. The experiments described below reveal expression of DR5 in tumor-associated endothelial cells (TECs), but not in normal endothelial cells. Treatment of syngeneic tumor-bearing mice with a crosslinked form of Apo2L/TRAIL led to a rapid collapse of the tumor vasculature; both the timing and appearance of this response were consistent with direct vascular disruption. Apoptotic markers appeared in TECs as early as two hours after DR5 ligation, followed by extensive tumor microhemorrhage. Vascular disruption required DR5 expression on TECs but not in the malignant tumor-cell compartment, and supported substantial anti-tumor efficacy even in the absence of direct DR5-mediated apoptosis in malignant cells. The experimental data thus suggest using proapoptotic receptor agonists as tumor-selective vascular disruption agents for cancer therapy.

To date, the therapeutic use for PARAs as anti-cancer agents has been predominantly based on the ability of PARAs to induce cancer-cell apoptosis via DR5 and/or DR4 (Johnstone et al., Nat. Rev. Cancer, 8:782-798 (2008); Ashkenazi et al., Nat. Rev. Drug Disc., 7:1001-1012 (2008)). However, some cancer cells remain refractory to death receptor ligation, suggesting that mechanisms of apoptosis evasion in malignant cells may limit clinical benefit of these agents (Yang et al., Curr. Opin. Cell Biol., (2010)). As disclosed in the present application, agents such as Apo2L/TRAIL can achieve anti-cancer efficacy by directly targeting the tumor vasculature. Importantly, DR5-mediated vascular disruption can exert tumoricidal activity even in the absence of DR5 function in malignant cells, highlighting the potential for inhibiting growth of tumors that otherwise would be expected to resist PARA-based therapy. Various vascular disrupting agents are in clinical development for cancer treatment; however, the therapeutic window for these agents might be limited by adverse events (Heath et al., Nat. Rev. Clin. Oncol., 6:395-404 (2009); McKeage et al., Cancer, 116:1859-1871 (2010)). Apo2L/TRAIL treatment was generally well-tolerated in the studies provided herein, consistent with the clinical safety profiles of PARAs to date (Ashkenazi et al., J. Clin. Invest., 118:1979-1990 (2008); Ashkenazi et al., Nat. Rev. Drug Discov., 7:1001-1012 (2008); Ashkenazi et al., Cytokine Growth Factor Rev., 19:325-331 (2008)). The PARAs may act as a unique class of tumor-selective vascular disruption agents, having the ability to treat tumors in which the malignant cell compartment is resistant to direct apoptosis induction.

Embodiments of the invention include compositions comprising a vascular disruption agent and uses of such agents to disrupt tumor vasculature. Optionally, the vascular disruption agent is an Apo2L/TRAIL polypeptide or death receptor agonist antibody.

Embodiments of the invention also include methods of vascular disruption in a mammalian tissue or cell sample, comprising steps of exposing said tissue or cell sample to an effective amount of Apo2L/TRAIL or death receptor agonist antibody. Optionally, the Apo2L/TRAIL polypeptide is a higher oligomeric form of Apo2L/TRAIL or cross-linked form of Apo2L/TRAIL.

Further methods of the invention include methods of treating cancer in a mammal, comprising administering an effective amount of Apo2L/TRAIL or death receptor agonist antibody to said mammal. Optionally, the methods comprise, in addition to administering an effective amount of Apo2L/TRAIL and/or death receptor agonist antibody, administering chemotherapeutic agent(s), radiation therapy, or other vascular inhibition therapy to said mammal. Optionally, the Apo2L/TRAIL polypeptide is a higher oligomeric form of Apo2L/TRAIL or cross-linked form of Apo2L/TRAIL.

The invention also provides uses of Apo2L/TRAIL or death receptor agonist antibody in the preparation of, or the manufacture of, a medicament for disrupting vasculature or for the treatment of cancer.

The invention further provides uses of Apo2L/TRAIL or death receptor agonist antibody in the manufacture of a kit for use in treating cancer.

Particular embodiments of the invention are further illustrated by the following claims:

1. A method of disrupting tumor associated vasculature in mammalian tissue or cells, comprising exposing said tissue or cells to a therapeutically effective amount of Apo2L/TRAIL polypeptide or death receptor agonist antibody.

2. The method of claim 1 wherein endothelial cells comprising the tumor associated vasculature express DR5 receptor.

3. The method of claim 1 wherein the mammalian tissue or cells comprise tumor or cancer cells that do not express DR5 receptor.

4. The method of claim 1 wherein the mammalian tissue or cells comprise tumor or cancer cells that express DR5 receptor and are resistant to apoptosis induction by said DR5 receptor.

5. The method of claim 1 wherein said Apo2L/TRAIL polypeptide is an oligomer or cross-linked form of Apo2L/TRAIL.

6. The method of claim 1 wherein said death receptor agonist antibody is an anti-DR5 monoclonal antibody.

7. A method of treating cancer in a mammal, comprising administering to said mammal a therapeutically effective amount of Apo2L/TRAIL polypeptide or death receptor agonist antibody to disrupt tumor associated vasculature in the mammal.

8. The method of claim 7 wherein said Apo2L/TRAIL polypeptide or death receptor agonist antibody disrupts said vasculature and inhibits blood flow to the tumor.

9. The method of claim 7 wherein endothelial cells comprising the tumor associated vasculature express DR5 receptor.

10. The method of claim 7 wherein the mammal's tumor or cancer cells do not express DR5 receptor.

11. The method of claim 7 wherein the mammal's tumor or cancer cells express DR5 receptor and are resistant to apoptosis induction by said DR5 receptor.

12. The method of claim 7 wherein one or more chemotherapeutic agents or radiation therapy is further administered to said mammal.

13. The method of claim 7 wherein anti-VEGF antibody is further administered to said mammal.

14. The method of claim 13 wherein said anti-VEGF antibody is bevacizumab.

15. The method of claim 7 wherein said Apo2L/TRAIL polypeptide is an oligomer or cross-linked form of Apo2L/TRAIL.

16. The method of claim 7 wherein said death receptor agonist antibody is an anti-DR5 monoclonal antibody.

17. The method of claim 7 wherein said cancer is lung carcinoma or pancreatic cancer.

18. Use of Apo2L/TRAIL polypeptide or death receptor agonist antibody in the manufacture of a medicament for disrupting tumor associated vasculature or for the treatment of cancer.

19. The use of claim 18 wherein said Apo2L/TRAIL polypeptide is an oligomer or cross-linked form of Apo2L/TRAIL.

20. The use of claim 18 wherein said death receptor agonist antibody is an anti-DR5 monoclonal antibody.

21. The use of Apo2L/TRAIL polypeptide or death receptor agonist antibody in the manufacture of a kit for use in treating cancer.

22. A kit for use in the treatment of cancer, comprising (a) a container comprising Apo2L/TRAIL polypeptide or death receptor agonist antibody and a pharmaceutically acceptable carrier or diluent within the container; and (b) a package insert with instructions for administering said Apo2L/TRAIL polypeptide or death receptor agonist antibody to disrupt tumor associated vasculature in a human patient having cancer.

### BRIEF DESCRIPTION OF THE DRAWINGS

FIG. 1 shows DR5-dependent disruption of the tumor vasculature by Apo2L/TRAIL. (a) Lewis lung carcinoma (LLC) tumors (˜500=³) grown in wildtype (DR5^{+/+}) or DR5-deficient (DR5^{-/-}) mice were dosed with an intraperitoneal (i.p.) injection of 10 mg/kg of Apo2L/TRAIL (consisting of 10 mg/kg of Flag-tagged Apo2L/TRAIL and 10 mg/kg anti-Flag antibody, given sequentially) or PBS. Tumors were examined macroscopically 24 hours after treatment for the appearance of vascular disruption. (b) Hematoxylin and eosin (H&E) staining of sections from LLC tumors grown in wildtype or DR5^{-/-} mice and treated with Apo2L/TRAIL. Images show extensive cell death and widespread hemorrhage in wildtype, but not DR5^{-/-}, mice treated with Apo2L/TRAIL. (c) Meca-32 staining was used to visualize the tumor endothelium; representative images showing disrupted blood vessels (arrows; upper right inset=enlarged image) in tumors from Apo2L/TRAIL-treated wildtype, but not DR5^{-/-} or untreated, mice. (d) LLC tumor-bearing wildtype or DR5^{-/-} mice (n=3-5/group) were treated with PBS or Apo2L/TRAIL. Two hours after treatment, mice were injected intravenously with the fluorescent blood pool probe AngioSense680IVM. Distribution of the fluorescent probe in the tumor was monitored at the indicated times on anesthetized mice. Error bars indicate the SEM. Data in FIG. 1 are representative of two or more independent experiments.

FIG. 2 shows DR5-mediated apoptosis in tumor-associated endothelial cells. (a) Analysis of DR5 expression by CD45^{low}CD31^{high} expressing tumor-associated endothelial cells (TECs) in LLC tumors grown in wildtype or DR5-deficient (DR5^{-/-}) mice. DR5 expression (shaded) versus isotype control (open) lines are shown from a pooled cell fraction generated from n=4 wildtype or DR5−/− LLC tumors. (b) Analysis of DR5 expression by CD45^{low}CD31^{high} expressing, “normal” kidney endothelial cells isolated from wildtype or DR5^{-/-} mice. Pooled kidney cell fractions were generated from the same mice that were analyzed in (a). (c) Immunohistochemical analysis of DR5 on LLC tumor sections. Red arrows highlight DR5 staining on endothelial (E) cells in tumors from wildtype but not DR5^{-/-}, mice. DR5-positive tumor (T) cells can be seen in both wildtype and DR5^{-/-} recipients (black arrows). (d) Meca-32 and activated caspase-3 (CC3) staining in serial LLC tumor sections collected from wildtype or DR5^{-/-} mice treated for 2 hours with Apo2L/TRAIL or PBS (control). Focal regions CC3-positive tumor cells (T, black arrows) can be seen in both untreated and Apo2L/TRAIL sections, but only Apo2L/TRAIL-treated tumors show evidence of apoptosis in vascular structures, revealed by Meca-32 staining (E, red arrows). (e) Quantitation of cleaved caspase-3 immunohistochemical staining on LL/C tumor sections from a time course of Apo2L/TRAIL treatment. The average of n=5 tumors for each time point is plotted; error bars indicate the SEM. Student's t-test was used to calculate statistical significance. (f) LLC tumors (>500=³) grown in wildtype or TNFR1/2-deficient (TNFR1/2^{-/-}) mice were dosed intraperitoneally with 10 mg/kg of Apo2L/TRAIL or PBS. Tumors were examined macroscopically 24 hours after treatment for the appearance of vascular disruption. (g) Table summarizing the incidence of vascular disruption in tumors grown in recipient mice with the indicated genotypes. Data in FIG. 2 are representative of two or more independent experiments.

FIG. 3 shows Apo2L/TRAIL effect on tumor vasculature is independent of tumor-cell DR5 expression. (a) Methylcholanthrene-induced (MCA) fibrosarcoma cell lines were derived from C57BL/6 wildtype (DR5^{+/+}) or DR5-deficient (DR5^{-/-}) mice and assayed for DR5 expression by flow cytometry. (b) Images of DR5^{+/+} or DR5^{-/-} fibrosarcoma tumors grown in C57BL/6 DR5^{+/+}Rag2^{-/-} (top and middle panels), or C57BL/6 DR5^{-/-} (bottom panels), recipients. Tumors were harvested at 24 hours post-treatment with Apo2L/TRAIL and compared with PBS-treated controls. (c) and (d) Apoptosis in tumor vasculature of MCA-induced tumors. DR5^{+/+} (c) or DR5^{-/-} (d) MCA-induced fibrosarcoma tumor cells were implanted in C57BL/6 DR5^{+/+}Rag2^{-/-} recipients and treated with Apo2L/TRAIL (10 mg/kg) for 4 hours. Serial sections from tumors were stained with antibodies specific for Meca-32 or active (cleaved) caspase-3 to localize endothelial and apoptotic cells, respectively. Data in FIG. 3 are representative of two or more independent experiments.

FIG. 4 shows vascular disruption by Apo2L/TRAIL contributes to anti-tumor efficacy in vivo. (a) DR5^{+/+} or DR5^{-/-} fibrosarcoma cell lines were treated in vitro with a dose titration of Apo2L/TRAIL. Caspase-8 and caspase 3/7 activity was quantified 4 hours after Apo2L/TRAIL treatment using luminescent substrate assays. Cell viability was determined 24 hr after Apo2L/TRAIL treatment using an ATP-based Cell Titer Glo assay. (b) DR5^{+/+} or DR5^{-/-} fibrosarcoma cell lines were grown in DR5^{+/+}Rag2^{-/-} recipient mice, treated with a single dose (10 mg/kg) of Apo2L/TRAIL, and harvested for immunohistochemical (IHC) staining with antibodies against active (cleaved) caspase-3. Graph shows quantitation of cleaved caspase-3 IHC staining on tumor sections from control (0 hr) or Apo2L/TRAIL-treated (24 hours) mice. The average of n=5 tumors for each group is plotted; error bars indicate the SEM. Student's t-test was used to calculate statistical significance. C57BL/6 DR5^{+/+}Rag2^{-/-} mice bearing wildtype (c) or DR5^{-/-} (d) MCA-induced tumors were treated with Apo2L/TRAIL five times per week for two weeks, and tumor growth was compared with untreated controls. Error bars indicate the SEM (n=8-10 mice/group). P-values were calculated using Student's t-test; asterisk indicates p<0.01; double asterisks indicate p<0.001. Data in FIG. 4 are representative of two or more independent experiments.

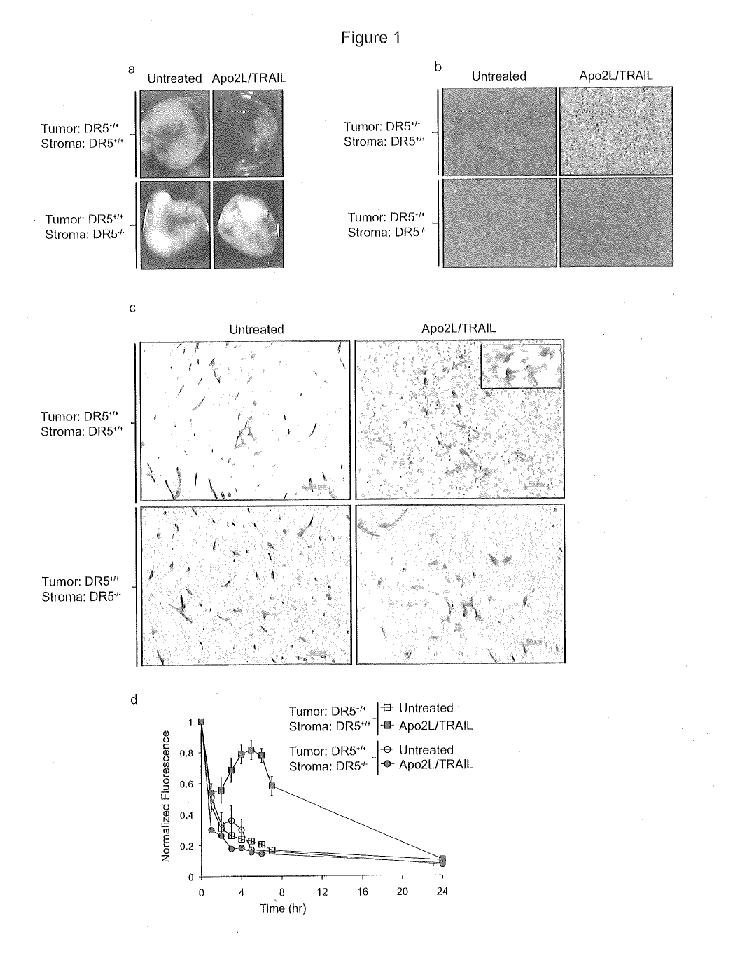

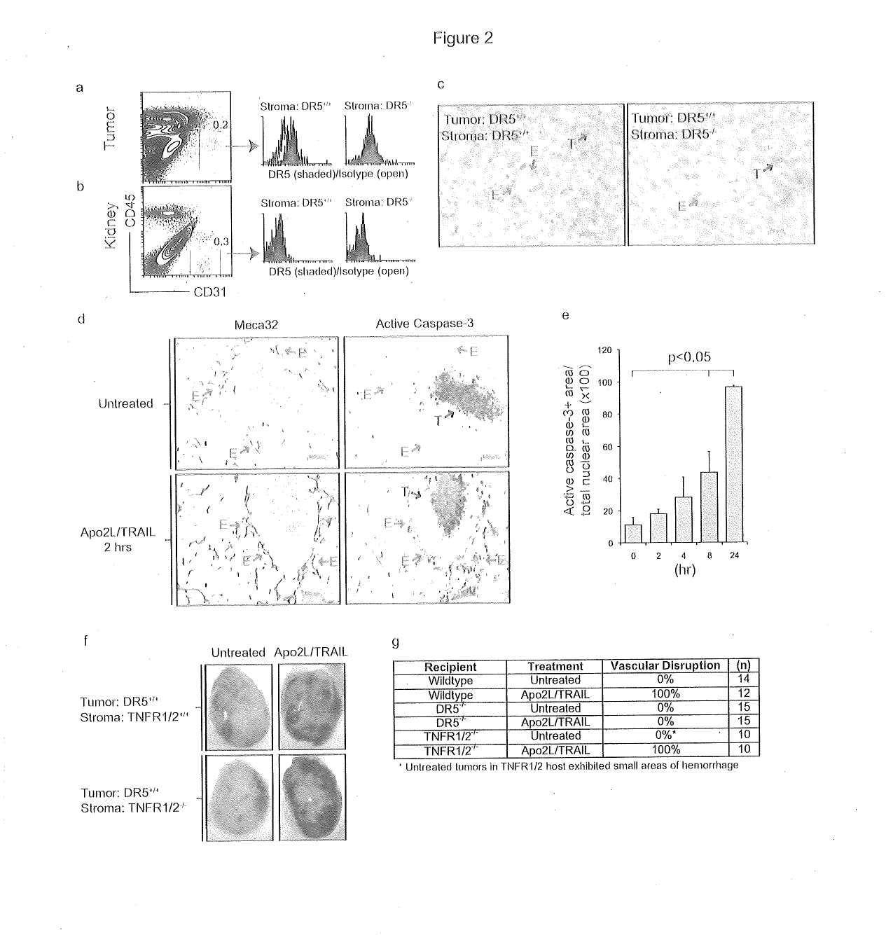

Supplementary FIG. 1 shows DR5 expression and sensitivity to Apo2L/TRAIL by murine tumor cell lines (obtained from American Type Culture Collection (ATCC)). (a) DR5 expression was assessed by flow cytomtery on B16 (melanoma), CT26 (colon carcinoma), 4T-1 (mammary carcinoma), EL4 (lymphoma), LLC (lung carcinoma) and Renca331 (renal cell carcinoma) cell lines. Profiles show DR5 expression (shaded lines) versus an isotype control antibody (open lines). (b) Renca331 and LLC (c) cells were treated with a dose titration of dulanermin or a Flag-tagged version of Apo2L/TRAIL combined with and anti-Flag cross-linking antibody. The fold-increase in caspase 3/7 activity and percent decrease in cell viability were quantified by the caspase-3/7 (4 hours) Glo or Cell Titer Glo (24 hours) assays (Promega). Data in Supplementary FIG. 1 are representative of two or more independent experiments

Supplementary FIG. 2 shows In vivo near infrared fluorescence imaging of Lewis lung carcinoma tumors. C57BL/6 wildtype (stroma DR5^{+/+}) or DR5-deficient (stroma DR5^{-/}) mice bearing LLC tumors were treated with Apo2L/TRAIL or PBS (control) 2 hours prior to injection of the fluorescent blood pool probe AngioSense680IVM. Shown are representative images from a time course following injection of the probe.

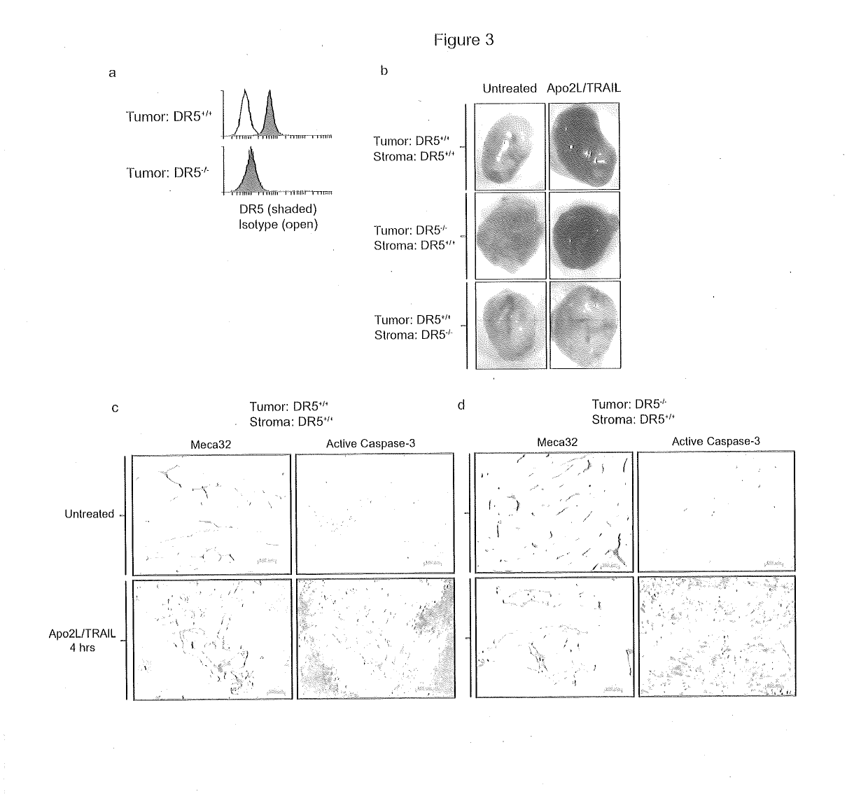

Supplementary FIG. 3 shows DR5 expression is expressed by LLC tumors grown in wildtype and DR5-deficient mice. Flow cytometry was used to evaluate DR5 surface expression ex vivo on tumor-associated leukocytes (CD45^{high}, fraction A) and LLC-enriched tumor cells (CD45^{low} CD31^{low}, fraction B) from tumors harvested from wildtype (DR5^{+/+}) or DR5-deficient (DR5^{-/-}) mice.

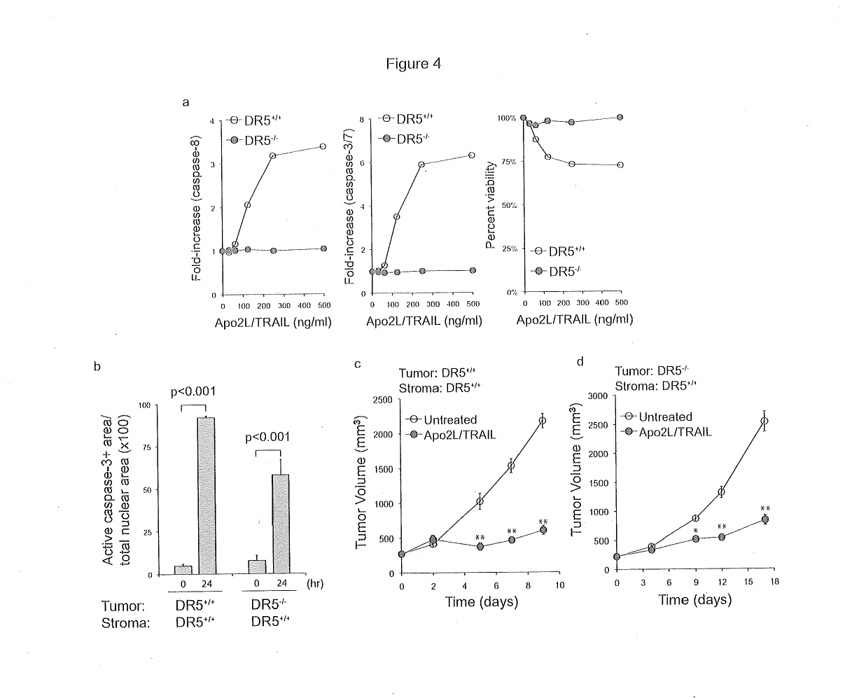

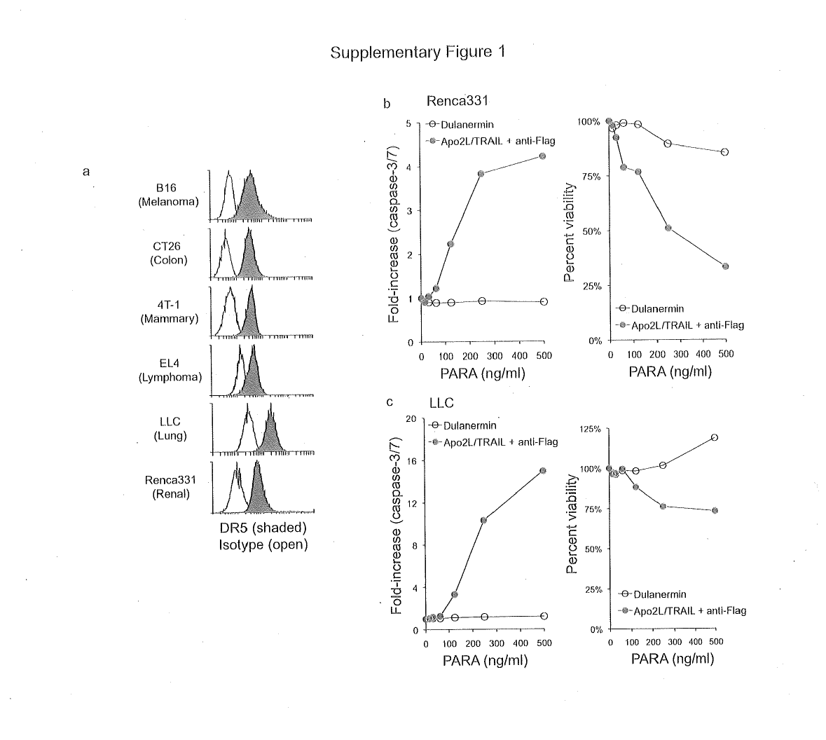

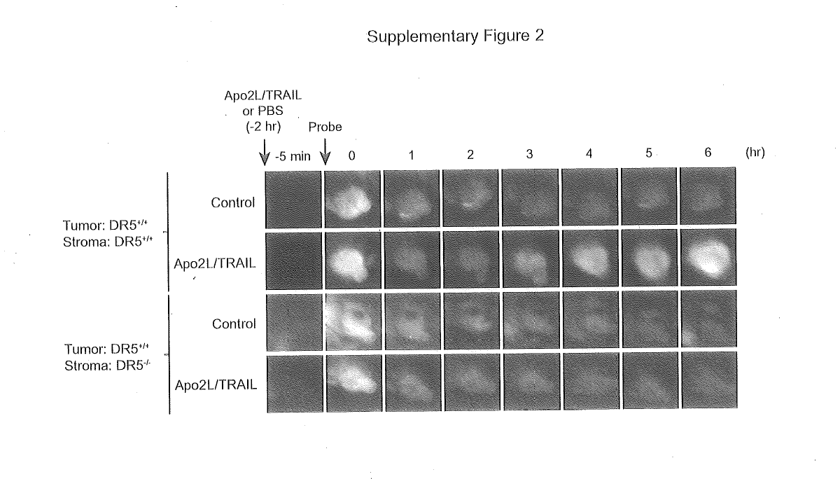

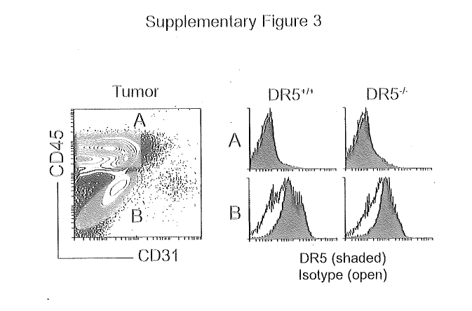

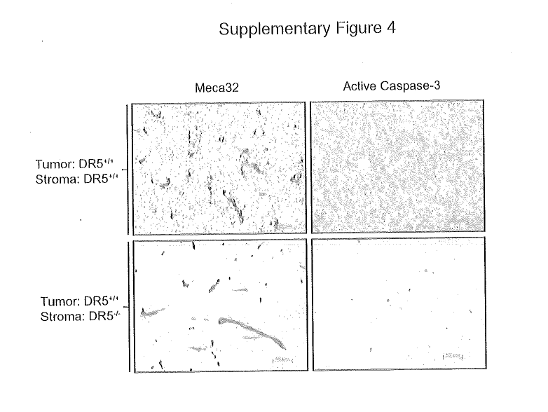

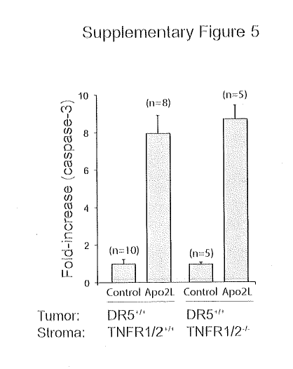

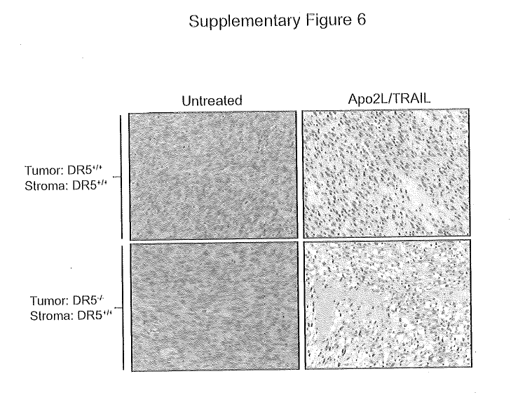

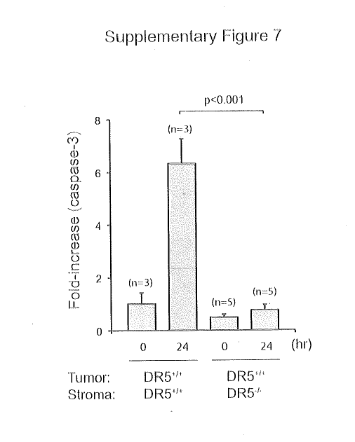

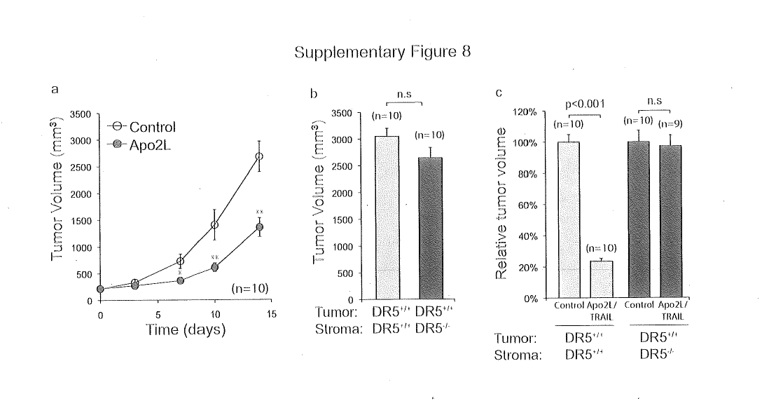

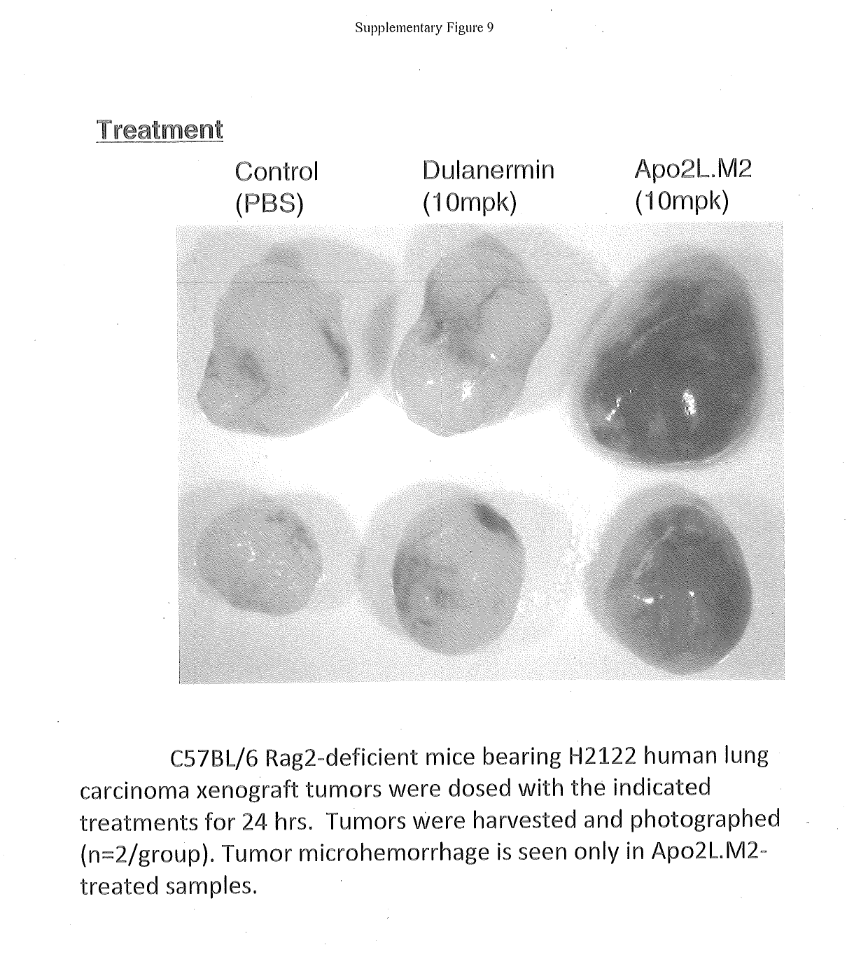

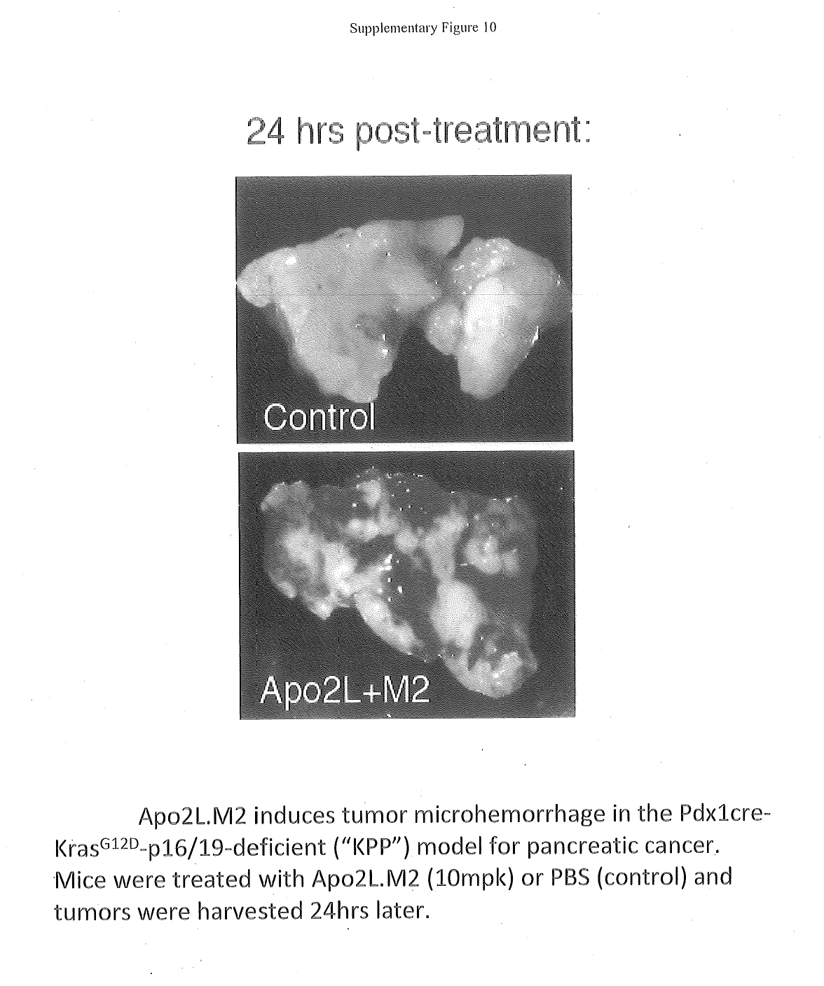

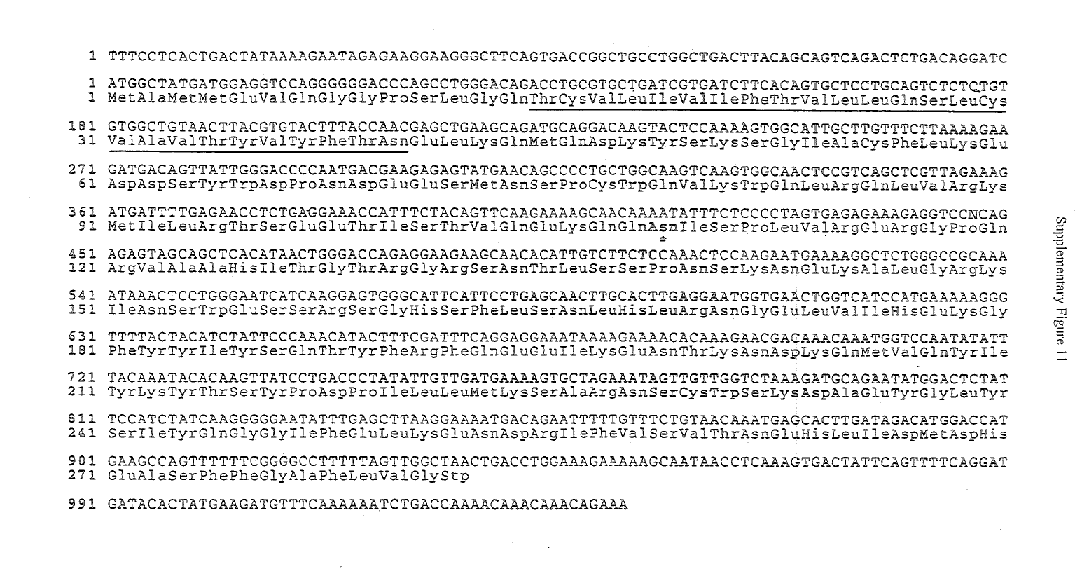

Supplementary FIG. 4 shows Apo2L/TRAIL induces apoptosis in LLC tumors grown in wildtype but not DR5-deficient mice. LLC tumor cells were implanted in C57BL/6 DR5^{+/+} or DR5^{-/-} recipients and treated with Apo2L/TRAIL (10 mg/kg) for 24 hours. Serial sections from treated tumors were stained with antibodies specific for Meca-32 or cleaved (active) caspase-3 to localize endothelial and apoptotic cells, respectively.

Supplementary FIG. 5 shows Apo2L/TRAIL induces tumor-cell apoptosis independent of TNFa signaling in the stroma. LLC tumor cells were implanted in C57BL/6 wildtype or TNFR1 and TNFR2 double-deficient (TNFR1/2^{-/-}) mice. After 24 hours treatment with Apo2L/TRAIL or PBS (control), tumors were harvested and flow cytometry was used to measure cleaved caspase-3 activity in tumor cells. Caspase-3 activity is represented as fold over control (PBS).

Supplementary FIG. 6 shows Apo2L/TRAIL induces hemorrhage in methylcholanthrene-induced (MCA) fibrosarcomas. H&E staining of sections from DR5^{+/+} or DR5^{-/-} MCA tumors grown in DR5^{+/+} Rag2^{-/-} mice and treated with Apo2L/TRAIL or PBS for 24 hours.

Supplementary FIG. 7 shows tumor-associated endothelial cell DR5 expression is required for Apo2L/TRAIL proapoptotic signaling in MCA-induced fibrosarcomas. Wildtype (DR5^{+/+}) MCA-induced fibrosarcoma cells were grown in C57BL/6 wildtype (DR5^{+/+}) or DR5^{-/-} mice. Tumors were harvested after 24 hr treatment with Apo2L/TRAIL and flow cytometry was used to measure cleaved caspase-3 activity in tumor cells. Caspase-3 activity is represented as fold-increase over control (0 hr).

Supplementary FIG. 8 shows tumor-associated endothelial cell DR5 expression is required for Apo2L/TRAIL anti-tumor activity in the LLC tumor model. (a) C57BL/6 mice bearing LLC tumors (<200=³) were treated with PBS (control) or Apo2L/TRAIL five times per week, for two weeks (n=10/group). Error bars indicate the SEM. (b) Day 12 tumor volumes of untreated LLC tumors implanted into C57B/L6 wildtype or DR5-deficient (DR5^{-/-}) mice (n=10/group). (c) LLC tumor cells grown in C57B/L6 wildtype or DR5^{-/-} mice were treated for five days with 10 mg/kg of Apo2L/TRAIL or PBS (control). Tumor volume relative to isotype control treated mice is indicated on the fifth day of treatment. Error bars indicate the SEM. Student's t-test was used to calculate statistical significance. Data in Supplementary FIG. 8 are representative of two or more independent experiments.

Supplementary FIG. 9 shows the effects of Dulanermin and Apo2L.M2 (cross-linked form of Apo2L) in mice bearing H2122 human lung carcinoma xenograft tumors.

Supplementary FIG. 10 shows the effects of Apo2L.M2 (cross-linked form of Apo2L) in a murine model of pancreatic cancer.

Supplementary FIG. 11 shows the encoding DNA (SEQ ID NO:2) and amino acid sequence (SEQ ID NO:1) for human Apo-2 ligand or TRAIL (“Apo2L/TRAIL”) polypeptide. The underlining in the Figure shows the predicted transmembrane region of the polypeptide. The sequence for human Apo2L/TRAIL polypeptide is also provided in WO97/01633 published Jan. 16, 1997 and WO97/25428 published Jul. 17, 1997.

### DETAILED DESCRIPTION OF THE PREFERRED EMBODIMENTS

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

It must be noted that as used herein and in the appended claims, the singular forms “a”, “and”, and “the” include plural referents unless the context clearly dictates otherwise.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

### Definitions

The terms “Apo-2 ligand”, “Apo-2L”, “Apo2L”, “Apo2L/TRAIL”, “Apo-2 ligand/TRAIL”, and “TRAIL” are used herein interchangeably to refer to a polypeptide sequence which includes amino acid residues 114-281, inclusive, 95-281, inclusive, residues 92-281, inclusive, residues 91-281, inclusive, residues 41-281, inclusive, residues 39-281, inclusive, residues 15-281, inclusive, or residues 1-281, inclusive, of the amino acid sequence shown in Supplementary FIG. 11, as well as biologically active fragments, deletional, insertional, or substitutional variants of the above sequences. In one embodiment, the polypeptide sequence comprises residues 114-281 of Supplementary FIG. 11. Optionally, the polypeptide sequence comprises residues 92-281 or residues 91-281 of Supplementary FIG. 11. The Apo-2L polypeptides may be encoded by the native nucleotide sequence shown in Supplementary FIG. 11. Optionally, the codon which encodes residue Proll9 (Supplementary FIG. 11) may be “CCT” or “CCG”. Optionally, the fragments or variants are biologically active and have at least about 80% amino acid sequence identity, more preferably at least about 90% sequence identity, and even more preferably, at least 95%, 96%, 97%, 98%, or 99% sequence identity with any one of the above sequences. The definition encompasses substitutional variants of Apo-2 ligand in which at least one of its native amino acids are substituted by another amino acid such as an alanine residue. Optional substitutional variants include one or more of the residue substitutions. Optional variants may comprise an amino acid sequence which differs from the native sequence Apo-2 ligand polypeptide sequence of Supplementary FIG. 11 and has one or more of the following amino acid substitutions at the residue position(s) in Supplementary FIG. 11: S96C; S101C; S111C; R170C; K179C. The definition also encompasses a native sequence Apo-2 ligand isolated from an Apo-2 ligand source or prepared by recombinant or synthetic methods. The Apo-2 ligand of the invention includes the polypeptides referred to as Apo-2 ligand or TRAIL disclosed in WO97/01633 published Jan. 16, 1997, WO97/25428 published Jul. 17, 1997, WO99/36535 published Jul. 22, 1999, WO 01/00832 published Jan. 4, 2001, WO02/09755 published Feb. 7, 2002, and WO 00/75191 published Dec. 14, 2000. The terms are used to refer generally to forms of the Apo-2 ligand which include monomer, dimer, trimer, hexamer or higher oligomer forms of the polypeptide. All numbering of amino acid residues referred to in the Apo-2L sequence use the numbering according to Supplementary FIG. 11, unless specifically stated otherwise. For instance, “D203” or “Asp203” refers to the aspartic acid residue at position 203 in the sequence provided in Supplementary FIG. 11.

A soluble form of recombinant human Apo2L/TRAIL polypeptide consisting of amino acids 114-281 of Supplementary FIG. 11 and produced in E. coli has been assigned the USAN name “Dulanermin” and references to “Dulanermin” refer to this form of Apo2L/TRAIL polypeptide. Dulanermin is manufactured and formulated by Genentech, Inc., South San Francisco, Calif. as described in WO 01/00832 published Jan. 4, 2001 and WO 03/042344 published May 22, 2003.

The term “Apo-2 ligand selective variant” as used herein refers to an Apo-2 ligand polypeptide which includes one or more amino acid mutations in a native Apo-2 ligand sequence and has selective binding affinity for either the DR4 receptor or the DR5 receptor. In one embodiment, the Apo-2 ligand variant has a selective binding affinity for the DR4 receptor and includes one or more amino acid substitutions in any one of positions 189, 191, 193, 199, 201 or 209 of a native Apo-2 ligand sequence. In another embodiment, the Apo-2 ligand variant has a selective binding affinity for the DR5 receptor and includes one or more amino acid substitutions in any one of positions 189, 191, 193, 264, 266, 267 or 269 of a native Apo-2 ligand sequence. Preferred Apo-2 ligand selective variants include one or more amino acid mutations and exhibit binding affinity to the DR4 receptor which is equal to or greater (≧) than the binding affinity of native sequence Apo-2 ligand to the DR4 receptor, and even more preferably, the Apo-2 ligand variants exhibit less binding affinity (<) to the DR5 receptor than the binding affinity exhibited by native sequence Apo-2 ligand to DRS. When binding affinity of such Apo-2 ligand variant to the DR4 receptor is approximately equal (unchanged) or greater than (increased) as compared to native sequence Apo-2 ligand, and the binding affinity of the Apo-2 ligand variant to the DR5 receptor is less than or nearly eliminated as compared to native sequence Apo-2 ligand, the binding affinity of the Apo-2 ligand variant, for purposes herein, is considered “selective” for the DR4 receptor. Preferred DR4 selective Apo-2 ligand variants of the invention will have at least 10-fold less binding affinity to DR5 receptor (as compared to native sequence Apo-2 ligand), and even more preferably, will have at least 100-fold less binding affinity to DR5 receptor (as compared to native sequence Apo-2 ligand). The respective binding affinity of the Apo-2 ligand variant may be determined and compared to the binding properties of native Apo-2L (such as the 114-281 form) by ELISA, RIA, and/or BIAcore assays, known in the art. Preferred DR4 selective Apo-2 ligand variants of the invention will induce apoptosis in at least one type of mammalian cell (preferably a cancer cell), and such apoptotic activity can be determined by known art methods such as the alamar blue or crystal violet assay. The DR4 selective Apo-2 ligand variants may or may not have altered binding affinities to any of the decoy receptors for Apo-2L, those decoy receptors being referred to in the art as DcR1, DcR2 and OPG.

Further preferred Apo-2 ligand selective variants include one or more amino acid mutations and exhibit binding affinity to the DR5 receptor which is equal to or greater (≧) than the binding affinity of native sequence Apo-2 ligand to the DR5 receptor, and even more preferably, such Apo-2 ligand variants exhibit less binding affinity (<) to the DR4 receptor than the binding affinity exhibited by native sequence Apo-2 ligand to DR4. When binding affinity of such Apo-2 ligand variant to the DR5 receptor is approximately equal (unchanged) or greater than (increased) as compared to native sequence Apo-2 ligand, and the binding affinity of the Apo-2 ligand variant to the DR4 receptor is less than or nearly eliminated as compared to native sequence Apo-2 ligand, the binding affinity of the Apo-2 ligand variant, for purposes herein, is considered “selective” for the DR5 receptor. Preferred DR5 selective Apo-2 ligand variants of the invention will have at least 10-fold less binding affinity to DR4 receptor (as compared to native sequence Apo-2 ligand), and even more preferably, will have at least 100-fold less binding affinity to DR4 receptor (as compared to native sequence Apo-2 ligand). The respective binding affinity of the Apo-2 ligand variant may be determined and compared to the binding properties of native Apo2L (such as the 114-281 form) by ELISA, RIA, and/or BIAcore assays, known in the art. Preferred DR5 selective Apo-2 ligand variants of the invention will induce apoptosis in at least one type of mammalian cell (preferably a cancer cell), and such apoptotic activity can be determined by known art methods such as the alamar blue or crystal violet assay. The DR5 selective Apo-2 ligand variants may or may not have altered binding affinities to any of the decoy receptors for Apo-2L, those decoy receptors being referred to in the art as DcR1, DcR2 and OPG.

Amino acid identification may use the single-letter alphabet or three-letter alphabet of amino acids, i.e.,

| | | | | | |
|---|---|---|---|---|---|
| Asp | D | Aspartic acid | Ile | I | Isoleucine |
| Thr | T | Threonine | Leu | L | Leucine |
| Ser | S | Serine | Tyr | Y | Tyrosine |
| Glu | E | Glutamic acid | Phe | F | Phenylalanine |
| Pro | P | Proline | His | H | Histidine |
| Gly | G | Glycine | Lys | K | Lysine |
| Ala | A | Alanine | Arg | R | Arginine |
| Cys | C | Cysteine | Trp | W | Tryptophan |
| Val | V | Valine | Gln | Q | Glutamine |
| Met | M | Methionine | Asn | N | Asparagine |

The term “Apo2L/TRAIL extracellular domain” or “Apo2L/TRAIL ECD” refers to a form of Apo2L/TRAIL which is essentially free of transmembrane and cytoplasmic domains. Ordinarily, the ECD will have less than 1% of such transmembrane and cytoplasmic domains, and preferably, will have less than 0.5% of such domains. It will be understood that any transmembrane domain(s) identified for the polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified. In preferred embodiments, the ECD will consist of a soluble, extracellular domain sequence of the polypeptide which is free of the transmembrane and cytoplasmic or intracellular domains (and is not membrane bound). Particular extracellular domain sequences of Apo-2L/TRAIL are described in PCT Publication Nos. WO97/01633 and WO97/25428.

The term “epitope tagged” when used herein refers to a chimeric polypeptide comprising Apo-2 ligand, or a portion thereof, fused to a “tag polypeptide”. The tag polypeptide has enough residues to provide an epitope against which an antibody can be made, yet is short enough such that it does not interfere with activity of the Apo-2 ligand. The tag polypeptide preferably also is fairly unique so that the antibody does not substantially cross-react with other epitopes. Suitable tag polypeptides generally have at least six amino acid residues and usually between about 8 to about 50 amino acid residues (preferably, between about 10 to about 20 residues).

The term “Apo2L/TRAIL monomer” or “Apo2L monomer” refers to a covalent chain of an extracellular domain sequence of Apo2L.

The term “Apo2L/TRAIL dimer” or “Apo2L dimer” refers to two Apo-2L monomers joined in a covalent linkage via a disulfide bond. The term as used herein includes free standing Apo2L dimers and Apo2L dimers that are within trimeric forms of Apo2L (i.e., associated with another, third Apo2L monomer).

The term “Apo2L/TRAIL trimer” or “Apo2L trimer” refers to three Apo2L monomers that are non-covalently associated.

Higher oligomeric forms of Apo2L/TRAIL, such as hexameric, nanomeric, and cross-linked forms of Apo2L/TRAIL are included for use in the invention. Determination of the presence and quantity of Apo2L/TRAIL monomer, dimer, or trimer (or other higher oligomeric forms) may be made using methods and assays known in the art (and using commercially available materials), such as native size exclusion HPLC (“SEC”), denaturing size exclusion using sodium dodecyl sulphate (“SDS-SEC”), reverse phase HPLC and capillary electrophoresis. Higher order oligomeric forms of Apo2L/TRAIL may be made using methods and materials known in the art, such as by using linkers or leucine zipper molecules.

“Apo-2 ligand receptor” includes the receptors referred to in the art as “DR4” and “DR5”. Pan et al. have described the TNF receptor family member referred to as “DR4” (Pan et al., Science, 276:111-113 (1997); see also WO98/32856 published Jul. 30, 1998; WO 99/37684 published Jul. 29, 1999; WO 00/73349 published Dec. 7, 2000; U.S. Pat. No. 6,433,147 issued Aug. 13, 2002; U.S. Pat. No. 6,461,823 issued Oct. 8, 2002, and U.S. Pat. No. 6,342,383 issued Jan. 29, 2002). Sheridan et al., Science, 277:818-821 (1997) and Pan et al., Science, 277:815-818 (1997) described another receptor for Apo2L/TRAIL (see also, WO98/51793 published Nov. 19, 1998; WO98/41629 published Sep. 24, 1998). This receptor is referred to as DR5 (the receptor has also been alternatively referred to as Apo-2; TRAIL-R, TR6, Tango-63, hAPO8, TRICK2 or KILLER; Screaton et al., Curr. Biol., 7:693-696 (1997); Walczak et al., EMBO J., 16:5386-5387 (1997); Wu et al., Nature Genetics, 17:141-143 (1997); WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999; US 2002/0072091 published Aug. 13, 2002; US 2002/0098550 published Dec. 7, 2001; U.S. Pat. No. 6,313,269 issued Dec. 6, 2001; US 2001/0010924 published Aug. 2, 2001; US 2003/01255540 published Jul. 3, 2003; US 2002/0160446 published Oct. 31, 2002, US 2002/0048785 published Apr. 25, 2002; U.S. Pat. No. 6,569,642 issued May 27, 2003, U.S. Pat. No. 6,072,047 issued Jun. 6, 2000, U.S. Pat. No. 6,642,358 issued Nov. 4, 2003). As described above, other receptors for Apo-2L include DcR1, DcR2, and OPG (see, Sheridan et al., supra; Marsters et al., supra; and Simonet et al., supra). The term “Apo-2L receptor” when used herein encompasses native sequence receptor and receptor variants. These terms encompass Apo-2L receptor expressed in a variety of mammals, including humans. Apo-2L receptor may be endogenously expressed as occurs naturally in a variety of human tissue lineages, or may be expressed by recombinant or synthetic methods. A “native sequence Apo-2L receptor” comprises a polypeptide having the same amino acid sequence as an Apo-2L receptor derived from nature. Thus, a native sequence Apo-2L receptor can have the amino acid sequence of naturally-occurring Apo-2L receptor from any mammal. Such native sequence Apo-2L receptor can be isolated from nature or can be produced by recombinant or synthetic means. The term “native sequence Apo-2L receptor” specifically encompasses naturally-occurring truncated or secreted forms of the receptor (e.g., a soluble form containing, for instance, an extracellular domain sequence), naturally-occurring variant forms (e.g., alternatively spliced forms) and naturally-occurring allelic variants. Receptor variants may include fragments or deletion mutants of the native sequence Apo-2L receptor. A transcriptional splice variant of human DR5 is known in the art. This DR5 splice variant encodes the 440 amino acid sequence of human DR5.

“Death receptor antibody” is used herein to refer generally to antibody or antibodies directed to a receptor in the tumor necrosis factor receptor superfamily and containing a death domain capable of signalling apoptosis, and such antibodies include DR5 antibody and DR4 antibody.

“DR5 receptor antibody”, “DR5 antibody”, or “anti-DR5 antibody” is used in a broad sense to refer to antibodies that bind to at least one form of a DR5 receptor, such as the 1-411 sequence or the 1-440 sequence, or extracellular domain thereof. Optionally the DR5 antibody is fused or linked to a heterologous sequence or molecule. Preferably the heterologous sequence allows or assists the antibody to form higher order or oligomeric complexes. Optionally, the DR5 antibody binds to DR5 receptor but does not bind or cross-react with any additional Apo-2L receptor (e.g. DR4, DcR1, or DcR2). Optionally the antibody is an agonist of DR5 signalling activity.

Optionally, the DR5 antibody of the invention binds to a DR5 receptor at a concentration range of about 0.1 nM to about 20 mM as measured in a BIAcore binding assay. Optionally, the DR5 antibodies of the invention exhibit an Ic 50 value of about 0.6 nM to about 18 mM as measured in a BIAcore binding assay.

“DR4 receptor antibody”, “DR4 antibody”, or “anti-DR4 antibody” is used in a broad sense to refer to antibodies that bind to at least one form of a DR4 receptor or extracellular domain thereof. Optionally the DR4 antibody is fused or linked to a heterologous sequence or molecule. Preferably the heterologous sequence allows or assists the antibody to form higher order or oligomeric complexes. Optionally, the DR4 antibody binds to DR4 receptor but does not bind or cross-react with any additional Apo-2L receptor (e.g. DR5, DcR1, or DcR2). Optionally the antibody is an agonist of DR4 signalling activity.

Optionally, the DR4 antibody of the invention binds to a DR4 receptor at a concentration range of about 0.1 nM to about 20 mM as measured in a BIAcore binding assay. Optionally, the DR4 antibodies of the invention exhibit an Ic 50 value of about 0.6 nM to about 18 mM as measured in a BIAcore binding assay.

The term “agonist” is used in the broadest sense, and includes any molecule that partially or fully enhances, stimulates or activates one or more biological activities of Apo2L/TRAIL, DR4 or DR5, in vitro, in situ, or in vivo. Examples of such biological activities are binding of Apo2L/TRAIL to DR4 or DR5, including apoptosis as well as those further reported in the literature. An agonist may function in a direct or indirect manner. For instance, the agonist may function to partially or fully enhance, stimulate or activate one or more biological activities of DR4 or DR5, in vitro, in situ, or in vivo as a result of its direct binding to DR4 or DR5, which causes receptor activation or signal transduction. The agonist may also function indirectly to partially or fully enhance, stimulate or activate one or more biological activities of DR4 or DR5, in vitro, in situ, or in vivo as a result of, e.g., stimulating another effector molecule which then causes DR4 or DR5 activation or signal transduction. It is contemplated that an agonist may act as an enhancer molecule which functions indirectly to enhance or increase DR4 or DR5 activation or activity. For instance, the agonist may enhance activity of endogenous Apo-2L in a mammal. This could be accomplished, for example, by pre-complexing DR4 or DR5 or by stabilizing complexes of the respective ligand with the DR4 or DR5 receptor (such as stabilizing native complex formed between Apo-2L and DR4 or DR5).

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols of the invention include those well known in the art and those publicly available, such as from commercially available sources.

The term “conjugate” is used herein according to its broadest definition to mean joined or linked together. Molecules are “conjugated” when they act or operate as if joined.

The term “extracellular domain” or “ECD” refers to a form of ligand or receptor which is essentially free of transmembrane and cytoplasmic domains. Ordinarily, the soluble ECD will have less than 1% of such transmembrane and cytoplasmic domains, and preferably, will have less than 0.5% of such domains.

The term “divalent metal ion” refers to a metal ion having two positive charges. Examples of divalent metal ions for use in the present invention include but are not limited to zinc, cobalt, nickel, cadmium, magnesium, and manganese. Particular forms of such metals that may be employed include salt forms (e.g., pharmaceutically acceptable salt forms), such as chloride, acetate, carbonate, citrate and sulfate forms of the above mentioned divalent metal ions. Divalent metal ions, as described herein, are preferably employed in concentrations or amounts (e.g., effective amounts) which are sufficient to, for example, (1) enhance storage stability of Apo-2L trimers over a desired period of time, (2) enhance production or yield of Apo-2L trimers in a recombinant cell culture or purification method, (3) enhance solubility (or reduce aggregation) of Apo-2L trimers, or (4) enhance Apo-2L trimer formation.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes. In preferred embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes protein in situ within recombinant cells, since at least one component of the protein's natural environment will not be present. Ordinarily, however, isolated protein will be prepared by at least one purification step.

An “isolated” nucleic acid molecule is a nucleic acid molecule that is identified and separated from at least one contaminant nucleic acid molecule with which it is ordinarily associated in the natural source of the nucleic acid. An isolated Apo-2 ligand nucleic acid molecule is other than in the form or setting in which it is found in nature. Isolated Apo-2 ligand nucleic acid molecules therefore are distinguished from the Apo-2 ligand nucleic acid molecule as it exists in natural cells. However, an isolated Apo-2 ligand nucleic acid molecule includes Apo-2 ligand nucleic acid molecules contained in cells that ordinarily express Apo-2 ligand where, for example, the nucleic acid molecule is in a chromosomal location different from that of natural cells.

“Percent (%) amino acid sequence identity” with respect to the sequences identified herein is defined as the percentage of amino acid residues in a candidate sequence that are identical with the amino acid residues in the Apo-2 ligand sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art can determine appropriate parameters for measuring alignment, including assigning algorithms needed to achieve maximal alignment over the full-length sequences being compared. For purposes herein, percent amino acid identity values can be obtained using the sequence comparison computer program, ALIGN-2, which was authored by Genentech, Inc. and the source code of which has been filed with user documentation in the US Copyright Office, Washington, D.C., 20559, registered under the US Copyright Registration No. TXU510087. The ALIGN-2 program is publicly available through Genentech, Inc., South San Francisco, Calif. All sequence comparison parameters are set by the ALIGN-2 program and do not vary.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “VEGF” or “VEGF-A” is used to refer to the 165-amino acid human vascular endothelial cell growth factor and related 121-, 189-, and 206-amino acid human vascular endothelial cell growth factors, as described by Leung et al. Science, 246:1306 (1989), and Houck et al. Mol. Endocrin., 5:1806 (1991), together with the naturally occurring allelic and processed forms thereof. VEGF-A is part of a gene family including VEGF-B, VEGF-C, VEGF-D, VEGF-E, VEGF-F, and P1GF. VEGF-A primarily binds to two high affinity receptor tyrosine kinases, VEGFR-1 (Flt-1) and VEGFR-2 (Flk-1/KDR), the latter being the major transmitter of vascular endothelial cell mitogenic signals of VEGF-A. Additionally, neuropilin-1 has been identified as a receptor for heparin-binding VEGF-A isoforms, and may play a role in vascular development. The term “VEGF” or “VEGF-A” also refers to VEGFs from non-human species such as mouse, rat, or primate. Sometimes the VEGF from a specific species is indicated by terms such as hVEGF for human VEGF or mVEGF for murine VEGF. The term “VEGF” is also used to refer to truncated forms or fragments of the polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF.

The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra.).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the p-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity.

In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

An antibody “which binds” an antigen of interest, e.g. VEGF, is one capable of binding that antigen with sufficient affinity and/or avidity, optionally such that the antibody is useful as a therapeutic agent for targeting a cell expressing the antigen.

An “anti-VEGF antibody” is an antibody that binds to VEGF with sufficient affinity and specificity. The antibody selected will normally have a sufficiently strong binding affinity for VEGF, for example, the antibody may bind hVEGF with a K_{d} value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g. RIA's), for example. Preferably, the anti-VEGF antibody of the invention can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as P1GF, PDGF or bFGF. Preferred anti-VEGF antibodies include a monoclonal antibody that binds to the same epitope as the monoclonal anti-VEGF antibody A4.6.1 produced by hybridoma ATCC HB 10709; a recombinant humanized anti-VEGF monoclonal antibody generated according to Presta et al. (1997) Cancer Res. 57:4593-4599, including but not limited to the antibody known as bevacizumab (BV; Avastin®). Bevacizumab includes mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005. Additional preferred antibodies include the G6 or B20 series antibodies (e.g., G6-31, B20-4.1), as described in PCT Application Publication No. WO2005/012359. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183 and Q89.

A “G6 series antibody” according to this disclosure is an anti-VEGF antibody that is derived from a sequence of a G6 antibody or G6-derived antibody according to any one of FIGS. 7, 24-26, and 34-35 of PCT Application Publication No. WO 2005/012359. In one preferred embodiment, the G6 series antibody binds to a functional epitope on human VEGF comprising residues F17, Y21, Q22, Y25, D63, 183 and Q89.

A “B20 series antibody” according to this disclosure is an anti-VEGF antibody that is derived from a sequence of the B20 antibody or a B20-derived antibody according to any one of FIGS. 27-29 of PCT Application Publication No. WO2005/012359. In one embodiment, the B20 series antibody binds to a functional epitope on human VEGF comprising residues F17, M18, D19, Y21, Y25, Q89, 191, K101, E103, and C104.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate”.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The homozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

“Complement dependent cytotoxicity” or “CDC” refer to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “therapeutically effective amount” refers to an amount of a therapeutic agent to treat or prevent a disease or disorder in a mammal. In the case of cancers, the therapeutically effective amount of the therapeutic agent may reduce the amount or extent of tumor vasculature, in particular, may reduce the amount or extent of tumor associated endothelial cells or tissue, reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, or fragments thereof.

The terms “vascular disrupting agent” or “VDA” refers in a broad sense to an agent which exhibits antivascular activity by disrupting established vasculature or blood vessels associated with a tumor or cancer tissue. Such disruption of the established vasculature can, for example, effect inhibition of tumor blood flow and/or necrosis or death of tumor or cancer cells or tissue.

The terms “apoptosis” and “apoptotic activity” are used in a broad sense and refer to the orderly or controlled form of cell death in mammals that is typically accompanied by one or more characteristic cell changes, including condensation of cytoplasm, loss of plasma membrane microvilli, segmentation of the nucleus, degradation of chromosomal DNA or loss of mitochondrial function. This activity can be determined and measured using well known art methods, for instance, by cell viability assays, FACS analysis or DNA electrophoresis, binding of annexin V, fragmentation of DNA, cell shrinkage, dilation of endoplasmic reticulum, cell fragmentation, and/or formation of membrane vesicles (called apoptotic bodies). Assays which determine the ability of an antibody (e.g. Rituximab) to induce apoptosis have been described in Shan et al. Cancer Immunol Immunther 48:673-83 (2000); Pedersen et al. Blood 99:1314-9 (2002); Demidem et al. Cancer Chemotherapy & Radiopharmaceuticals 12(3):177-186 (1997), for example.

The terms “cancer”, “cancerous”, “tumor” and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

The term “pre-cancerous” refers to a condition or a growth that typically precedes or develops into a cancer. A “pre-cancerous” growth will have cells that are characterized by abnormal cell cycle regulation, proliferation, or differentiation, which can be determined by markers of cell cycle regulation, cellular proliferation, or differentiation.

By “dysplasia” is meant any abnormal growth or development of tissue, organ, or cells. Preferably, the dysplasia is high grade or precancerous.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass.

Both stimulatory and inhibitory molecular pathways within the tumor cell regulate this behavior, and interactions between the tumor cell and host cells in the distant site are also significant.

By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer.

By “primary tumor” or “primary cancer” is meant the original cancer and not a metastatic lesion located in another tissue, organ, or location in the subject's body.

By “benign tumor” or “benign cancer” is meant a tumor that remains localized at the site of origin and does not have the capacity to infiltrate, invade, or metastasize to a distant site.

By “tumor burden” is meant the number of cancer cells, the size of a tumor, or the amount of cancer in the body. Tumor burden is also referred to as tumor load.

By “tumor number” is meant the number of tumors.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TP0); nerve growth factors; platelet-growth factor; transforming growth factors (TGFs) such as TGF-α and TGF-β; insulin-like growth factor-I and -II; erythropoietin (EPO); osteoinductive factors; interferons such as interferon-α, -β, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-2, IL-3, IL-4, IL-5, IL-6, IL-7, IL-8, IL-9, IL-11, IL-12; and other polypeptide factors including LIF and kit ligand (KL). As used herein, the term cytokine includes proteins from natural sources or from recombinant cell culture and biologically active equivalents of the native sequence cytokines.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products, etc.

The terms “treating”, “treatment” and “therapy” as used herein refer to curative therapy, prophylactic therapy, and preventative therapy.

The term “mammal” as used herein refers to any mammal classified as a mammal, including humans, cows, horses, dogs and cats. In a preferred embodiment of the invention, the mammal is a human.

II. Compositions and Methods of the Invention

A cytokine related to the TNF ligand family, the cytokine identified herein as “Apo-2 ligand” or “TRAIL” has been described. The predicted mature amino acid sequence of native human Apo-2 ligand contains 281 amino acids, and has a calculated molecular weight of approximately 32.5 kDa. The absence of a signal sequence and the presence of an internal hydrophobic region suggest that Apo-2 ligand is a type II transmembrane protein. Soluble extracellular domain Apo-2 ligand polypeptides have also been described. See, e.g., WO97/25428 published Jul. 17, 1997. Apo-2L substitutional variants have further been described. Alanine scanning techniques have been utilized to identify various substitutional variant molecules having biological activity. Particular substitutional variants of the Apo-2 ligand include those in which at least one amino acid is substituted by another amino acid such as an alanine residue. These substitutional variants are identified, for example, as “D203A”; “D218A” and “D269A.” This nomenclature is used to identify Apo-2 ligand variants wherein the aspartic acid residues at positions 203, 218, and/or 269 (using the numbering shown in Supplementary FIG. 11) are substituted by alanine residues. Optionally, the Apo-2L variants of the present invention may comprise one or more of the amino acid substitutions. Optionally, such Apo-2L variants will be DR4 or DR5 receptor selective variants.

The description below relates to methods of producing Apo-2 ligand, including Apo-2 ligand variants, by culturing host cells transformed or transfected with a vector containing Apo-2 ligand encoding nucleic acid and recovering the polypeptide from the cell culture.

The DNA encoding Apo-2 ligand may be obtained from any cDNA library prepared from tissue believed to possess the Apo-2 ligand mRNA and to express it at a detectable level. Accordingly, human Apo-2 ligand DNA can be conveniently obtained from a cDNA library prepared from human tissues, such as the bacteriophage library of human placental cDNA as described in WO97/25428. The Apo-2 ligand-encoding gene may also be obtained from a genomic library or by oligonucleotide synthesis.

Libraries can be screened with probes (such as antibodies to the Apo-2 ligand or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding Apo-2 ligand is to use PCR methodology [Sambrook et al., supra; Dieffenbach et al., PCR Primer:A Laboratory Manual (Cold Spring Harbor Laboratory Press, 1995)].

Amino acid sequence fragments or variants of Apo-2 ligand can be prepared by introducing appropriate nucleotide changes into the Apo-2 ligand DNA, or by synthesis of the desired Apo-2 ligand polypeptide. Such fragments or variants represent insertions, substitutions, and/or deletions of residues within or at one or both of the ends of the intracellular region, the transmembrane region, or the extracellular region, or of the amino acid sequence shown for the full-length Apo-2 ligand shown in Supplementary FIG. 11. Any combination of insertion, substitution, and/or deletion can be made to arrive at the final construct, provided that the final construct possesses, for instance, a desired biological activity, such as apoptotic activity, as defined herein. In a preferred embodiment, the fragments or variants have at least about 80% amino acid sequence identity, more preferably, at least about 90% sequence identity, and even more preferably, at least 95%, 96%, 97%, 98% or 99% sequence identity with the sequences identified herein for the intracellular, transmembrane, or extracellular domains of Apo-2 ligand, or the full-length sequence for Apo-ligand. The amino acid changes also may alter post-translational processes of the Apo-2 ligand, such as changing the number or position of glycosylation sites or altering the membrane anchoring characteristics.

Variations in the Apo-2 ligand sequence as described above can be made using any of the techniques and guidelines for conservative and non-conservative mutations set forth in U.S. Pat. No. 5,364,934. These include oligonucleotide-mediated (site-directed) mutagenesis, alanine scanning, and PCR mutagenesis.

Scanning amino acid analysis can be employed to identify one or more amino acids along a contiguous sequence. Among the preferred scanning amino acids are relatively small, neutral amino acids. Such amino acids include alanine, glycine, serine and cysteine. Alanine is typically a preferred scanning amino acid among this group because it eliminates the side-chain beyond the beta-carbon and is less likely to alter the main-chain conformation of the variant. [Cunningham et al., Science, 244:1081 (1989)]. Alanine is also typically preferred because it is the most common amino acid. Further, it is frequently found in both buried and exposed positions [Creighton, The Proteins, (W.H. Freeman & Co., NY); Chothia, J. Mol. Biol., 150:1 (1976)].

Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)
(2) uncharged polar: Gly (G), Ser (S), Thr (T), Cys (C), Tyr (Y), Asn (N), Gln (Q)
(3) acidic: Asp (D), Glu (E)
(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Variations in the Apo-2 ligand sequence also included within the scope of the invention relate to amino-terminal derivatives or modified forms. Such Apo-2 ligand sequences include any of the Apo-2 ligand polypeptides described herein having a methionine or modified methionine (such as formyl methionyl or other blocked methionyl species) at the N-terminus of the polypeptide sequence.

The nucleic acid (e.g., cDNA or genomic DNA) encoding native or variant Apo-2 ligand may be inserted into a replicable vector for further cloning (amplification of the DNA) or for expression. Various vectors are publicly available. The vector components generally include, but are not limited to, one or more of the following: a signal sequence, an origin of replication, one or more marker genes, an enhancer element, a promoter, and a transcription termination sequence, each of which is described below. Optional signal sequences, origins of replication, marker genes, enhancer elements and transcription terminator sequences that may be employed are known in the art and described in further detail in WO97/25428.

Expression and cloning vectors usually contain a promoter that is recognized by the host organism and is operably linked to the Apo-2 ligand nucleic acid sequence. Promoters are untranslated sequences located upstream (5′) to the start codon of a structural gene (generally within about 100 to 1000 bp) that control the transcription and translation of a particular nucleic acid sequence, such as the Apo-2 ligand nucleic acid sequence, to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. At this time a large number of promoters recognized by a variety of potential host cells are well known. These promoters are operably linked to Apo-2 ligand encoding DNA by removing the promoter from the source DNA by restriction enzyme digestion and inserting the isolated promoter sequence into the vector. Both the native Apo-2 ligand promoter sequence and many heterologous promoters may be used to direct amplification and/or expression of the Apo-2 ligand DNA.

Promoters suitable for use with prokaryotic and eukaryotic hosts are known in the art, and are described in further detail in WO97/25428.

A preferred method for the production of soluble Apo-2L in E. coli employs an inducible promoter for the regulation of product expression. The use of a controllable, inducible promoter allows for culture growth to the desirable cell density before induction of product expression and accumulation of significant amounts of product which may not be well tolerated by the host.

Several inducible promoter systems (T7 polymerase, trp and alkaline phosphatase (AP)) have been evaluated by Applicants for the expression of Apo-2L (form 114-281). The use of each of these three promoters resulted in significant amounts of soluble, biologically active Apo-2L trimer being recovered from the harvested cell paste. The AP promoter is preferred among these three inducible promoter systems tested because of tighter promoter control and the higher cell density and titers reached in harvested cell paste.

Construction of suitable vectors containing one or more of the above-listed components employs standard ligation techniques. Isolated plasmids or DNA fragments are cleaved, tailored, and re-ligated in the form desired to generate the plasmids required.

For analysis to confirm correct sequences in plasmids constructed, the ligation mixtures can be used to transform E. coli K12 strain 294 (ATCC 31,446) and successful transformants selected by ampicillin or tetracycline resistance where appropriate. Plasmids from the transformants are prepared, analyzed by restriction endonuclease digestion, and/or sequenced using standard techniques known in the art. [See, e.g., Messing et al., Nucleic Acids Res., 9:309 (1981); Maxam et al., Methods in Enzymology, 65:499 (1980)].

Expression vectors that provide for the transient expression in mammalian cells of DNA encoding Apo-2 ligand may be employed. In general, transient expression involves the use of an expression vector that is able to replicate efficiently in a host cell, such that the host cell accumulates many copies of the expression vector and, in turn, synthesizes high levels of a desired polypeptide encoded by the expression vector

[Sambrook et al., supra]. Transient expression systems, comprising a suitable expression vector and a host cell, allow for the convenient positive identification of polypeptides encoded by cloned DNAs, as well as for the rapid screening of such polypeptides for desired biological or physiological properties. Thus, transient expression systems are particularly useful in the invention for purposes of identifying analogs and variants of Apo-2 ligand that are biologically active Apo-2 ligand.

Other methods, vectors, and host cells suitable for adaptation to the synthesis of Apo-2 ligand in recombinant vertebrate cell culture are described in Gething et al., Nature, 293:620-625 (1981); Mantei et al., Nature, 281:40-46 (1979); EP 117,060; and EP 117,058.

Suitable host cells for cloning or expressing the DNA in the vectors herein include prokaryote, yeast, or higher eukaryote cells. Suitable prokaryotes for this purpose include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Preferably, the host cell should secrete minimal amounts of proteolytic enzymes.

In addition to prokaryotes, eukaryotic microbes such as filamentous fungi or yeast are suitable cloning or expression hosts for Apo-2 ligand-encoding vectors. Suitable host cells for the expression of glycosylated Apo-2 ligand are derived from multicellular organisms. Examples of all such host cells, including CHO cells, are described further in WO97/25428.

Host cells are transfected and preferably transformed with the above-described expression or cloning vectors for Apo-2 ligand production and cultured in nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of this vector occurs within the host cell.

Transformation means introducing DNA into an organism so that the DNA is replicable, either as an extrachromosomal element or by chromosomal integrant. Depending on the host cell used, transformation is done using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. In addition, plants may be transfected using ultrasound treatment as described in WO 91/00358 published 10 Jan. 1991.

For mammalian cells without such cell walls, the calcium phosphate precipitation method of Graham and van der Eb, Virology, 52:456-457 (1978) may be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Prokaryotic cells used to produce Apo-2 ligand may be cultured in suitable culture media as described generally in Sambrook et al., supra. Particular forms of culture media that may be employed for culturing E. coli are described in the literature. Mammalian host cells used to produce Apo-2 ligand may be cultured in a variety of culture media.

Examples of commercially available culture media include Ham's F10 (Sigma), Minimal Essential Medium (“MEM”, Sigma), RPMI-1640 (Sigma), and Dulbecco's Modified Eagle's Medium (“DMEM”, Sigma). Any such media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

In general, principles, protocols, and practical techniques for maximizing the productivity of mammalian cell cultures can be found in Mammalian Cell Biotechnology: A Practical Approach, M. Butler, ed. (IRL Press, 1991).

In accordance with one aspect of the present invention, one or more divalent metal ions will typically be added to or included in the culture media for culturing or fermenting the host cells. The divalent metal ions are preferably present in or added to the culture media at a concentration level sufficient to enhance storage stability, enhance solubility, or assist in forming stable Apo-2L trimers coordinated by one or more zinc ions. The amount of divalent metal ions which may be added will be dependent, in part, on the host cell density in the culture or potential host cell sensitivity to such divalent metal ions. At higher host cell densities in the culture, it may be beneficial to increase the concentration of divalent metal ions. If the divalent metal ions are added during or after product expression by the host cells, it may be desirable to adjust or increase the divalent metal ion concentration as product expression by the host cells increases. It is generally believed that trace levels of divalent metal ions which may be present in typical commonly available cell culture media may not be sufficient for stable trimer formation. Thus, addition of further quantities of divalent metal ions, as described herein, is preferred.

The divalent metal ions are preferably added to the culture media at a concentration which does not adversely or negatively affect host cell growth, if the divalent metal ions are being added during the growth phase of the host cells in the culture. In shake flask cultures, it was observed that ZnSO₄ added at concentrations of greater than 1 mM can result in lower host cell density. Those skilled in the art appreciate that bacterial cells can sequester metal ions effectively by forming metal ion complexes with cellular matrices. Thus, in the cell cultures, it is preferable to add the selected divalent metal ions to the culture media after the growth phase (after the desired host cell density is achieved) or just prior to product expression by the host cells. To ensure that sufficient amounts of divalent metal ions are present, additional divalent metal ions may be added or fed to the cell culture media during the product expression phase.

The divalent metal ion concentration in the culture media should not exceed the concentration which may be detrimental or toxic to the host cells. In the methods employing the host cell, E. coli, it is preferred that the concentration of the divalent metal ion concentration in the culture media does not exceed about 1 mM (preferably, <1 mM). Even more preferably, the divalent metal ion concentration in the culture media is about 50 micromolar to about 250 micromolar. Most preferably, the divalent metal ion used in such methods is zinc sulfate. It is desirable to add the divalent metal ions to the cell culture in an amount wherein the metal ions and Apo-2 ligand trimer can be present at a one to one molar ratio.

The divalent metal ions can be added to the cell culture in any acceptable form. For instance, a solution of the metal ion can be made using water, and the divalent metal ion solution can then be added or fed to the culture media.

Expression of the Apo-2L may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Various labels may be employed, most commonly radioisotopes, and particularly ³²P. However, other techniques may also be employed, such as using biotin-modified nucleotides for introduction into a polynucleotide. The biotin then serves as the site for binding to avidin or antibodies, which may be labeled with a wide variety of labels, such as radionucleotides, fluorescers or enzymes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected. Gene expression, alternatively, may be measured by immunological methods, such as immunohistochemical staining of cells or tissue sections and assay of cell culture or body fluids, to quantitate directly the expression of gene product. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like.

Antibodies useful for immunohistochemical staining and/or assay of sample fluids may be either monoclonal or polyclonal, and may be prepared in any mammal. Conveniently, the antibodies may be prepared against a native Apo-2 ligand polypeptide or against a synthetic peptide based on the DNA sequences provided herein or against exogenous sequence fused to Apo-2 ligand DNA and encoding a specific antibody epitope.

Apo-2 ligand preferably is recovered from the culture medium as a secreted polypeptide, although it also may be recovered from host cell lysates when directly produced without a secretory signal. If the Apo-2 ligand is membrane-bound, it can be released from the membrane using a suitable detergent solution (e.g. Triton-X 100) or its extracellular region may be released by enzymatic cleavage.

When Apo-2 ligand is produced in a recombinant cell other than one of human origin, the Apo-2 ligand is free of proteins or polypeptides of human origin. However, it is usually necessary to recover or purify Apo-2 ligand from recombinant cell proteins or polypeptides to obtain preparations that are substantially homogeneous as to Apo-2 ligand. As a first step, the culture medium or lysate may be centrifuged to remove particulate cell debris. Apo-2 ligand thereafter is purified from contaminant soluble proteins and polypeptides, with the following procedures being exemplary of suitable purification procedures: by fractionation on an ion-exchange column; ethanol precipitation; reverse phase HPLC; chromatography on silica or on a cation-exchange resin such as DEAE or CM; chromatofocusing; SDS-PAGE; ammonium sulfate precipitation; gel filtration using, for example, Sephadex G-75; diafiltration and protein A Sepharose columns to remove contaminants such as IgG.

In a preferred embodiment, the Apo-2 ligand can be isolated by affinity chromatography. Apo-2 ligand fragments or variants in which residues have been deleted, inserted, or substituted are recovered in the same fashion as native Apo-2 ligand, taking account of any substantial changes in properties occasioned by the variation. For example, preparation of an Apo-2 ligand fusion with another protein or polypeptide, e.g., a bacterial or viral antigen, facilitates purification; an immunoaffinity column containing antibody to the antigen can be used to adsorb the fusion polypeptide.

A protease inhibitor such as phenyl methyl sulfonyl fluoride (PMSF) also may be useful to inhibit proteolytic degradation during purification, and antibiotics may be included to prevent the growth of adventitious contaminants. One skilled in the art will appreciate that purification methods suitable for native Apo-2 ligand may require modification to account for changes in the character of Apo-2 ligand or its variants upon expression in recombinant cell culture.

During any such purification steps, it may be desirable to expose the recovered Apo-2L to a divalent metal ion-containing solution or to purification material (such as a chromatography medium or support) containing one or more divalent metal ions. In a preferred embodiment, the divalent metal ions and/or reducing agent is used during recovery or purification of the Apo-2L. Optionally, both divalent metal ions and reducing agent, such as DTT or BME, may be used during recovery or purification of the Apo-2L. It is believed that use of divalent metal ions during recovery or purification will provide for stability of Apo-2L trimer or preserve Apo-2L trimer formed during the cell culturing step.

The description below also relates to methods of producing Apo-2 ligand covalently attached (hereinafter “conjugated”) to one or more chemical groups. Chemical groups suitable for use in an Apo-2L conjugate of the present invention are preferably not significantly toxic or immunogenic. The chemical group is optionally selected to produce an Apo-2L conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to polypeptides such as an Apo-2L at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of the Apo-2L can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are nonhomogeneous preparations sold according to average molecular weight. For example, commercially available PEG(5000) preparations typically contain molecules that vary slightly in molecular weight, usually ±500 D.

The Apo-2 ligand of the invention may be in various forms, such as in monomer form or trimer form (comprising three monomers). Optionally, an Apo-2L trimer will be pegylated in a manner such that a PEG molecule is linked or conjugated to one, two or each of the three monomers that make up the trimeric Apo-2L. In such an embodiment, it is preferred that the PEG employed have an average molecular weight of about 1,000 to about 5,000 D. It is also contemplated that the Apo-2L trimers may be “partially” pegylated, i.e., wherein only one or two of the three monomers that make up the trimer are linked or conjugated to PEG.

A variety of methods for pegylating proteins are known in the art. Specific methods of producing proteins conjugated to PEG include the methods described in U.S. Pat. No. 4,179,337, U.S. Pat. No. 4,935,465 and U.S. Pat. No. 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

It is further contemplated that the Apo2L described herein may be also be linked or cross-linked with tag molecules or leucine zipper sequences using techniques known in the art. Thus, the Apo-2 ligand may be fused to another, heterologous polypeptide. In one embodiment, the chimeric polypeptide comprises a fusion of the Apo-2 ligand with a tag polypeptide which provides an epitope to which an anti-tag antibody can selectively bind. The epitope tag is generally placed at the amino- or carboxyl-terminus of the Apo-2 ligand. The presence of such epitope-tagged forms of the Apo-2 ligand can be detected using an antibody against the tag polypeptide. Also, provision of the epitope tag enables the Apo-2 ligand to be readily purified by affinity purification using an anti-tag antibody or another type of affinity matrix that binds to the epitope tag.

Various tag polypeptides and their respective antibodies are well known in the art. Examples include the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)]. Once the tag polypeptide has been selected, an antibody thereto can be generated using the techniques disclosed herein.

Generally, epitope-tagged Apo-2 ligand may be constructed and produced according to the methods described above for native and variant Apo-2 ligand. Apo-2 ligand-tag polypeptide fusions are preferably constructed by fusing the cDNA sequence encoding the Apo-2 ligand portion in-frame to the tag polypeptide DNA sequence and expressing the resultant DNA fusion construct in appropriate host cells. Ordinarily, when preparing the Apo-2 ligand-tag polypeptide chimeras of the present invention, nucleic acid encoding the Apo-2 ligand will be fused at its 3′ end to nucleic acid encoding the N-terminus of the tag polypeptide, however 5′ fusions are also possible. An example of epitope-tagged Apo-2 ligand is described in further detail in the Examples below.

Epitope-tagged Apo-2 ligand can be purified by affinity chromatography using the anti-tag antibody. The matrix to which the affinity antibody is attached may include, for instance, agarose, controlled pore glass or poly(styrenedivinyl)benzene). The epitope-tagged Apo-2 ligand can then be eluted from the affinity column using techniques known in the art.

Formulations comprising Apo2L/TRAIL are also provided by the present invention. It is believed that such formulations will be particularly suitable for storage as well as for therapeutic administration. The formulations may be prepared by known techniques. For instance, the formulations may be prepared by buffer exchange on a gel filtration column.

Formulations comprising Apo2L/TRAIL are also provided by the present invention. It is believed that such formulations will be particularly suitable for storage as well as for therapeutic administration. The formulations may be prepared by known techniques.

Typically, an appropriate amount of an acceptable salt or carrier is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of agent.

Therapeutic compositions can be prepared by mixing the desired molecules having the appropriate degree of purity with optional carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Diagnosis in mammals of the various pathological conditions described herein can be made by the skilled practitioner. Diagnostic techniques are available in the art which allow, e.g., for the diagnosis or detection of cancer in a mammal. For instance, cancers may be identified through techniques, including but not limited to, palpation, blood analysis, x-ray, NMR and the like. Cancer staging systems describe how far the cancer has spread anatomically and attempt to put patients with similar prognosis and treatment in the same staging group. Several tests may be performed to help stage cancer including biopsy and certain imaging tests such as a chest x-ray, mammogram, bone scan, CT scan, and MRI scan. Blood tests and a clinical evaluation are also used to evaluate a patient's overall health and detect whether the cancer has spread to certain organs.

The tumor can be a solid tumor. A solid tumor includes any cancer of body tissues other than blood, bone marrow, or the lymphatic system. Solid tumors can be further divided into those of epithelial cell origin and those of non-epithelial cell origin. Examples of epithelial cell solid tumors include tumors of the gastrointestinal tract, colon, breast, prostate, lung, kidney, liver, pancreas, ovary, head and neck, oral cavity, stomach, duodenum, small intestine, large intestine, anus, gall bladder, labium, nasopharynx, skin, uterus, male genital organ, urinary organs, bladder, and skin. Solid tumors of non-epithelial origin include sarcomas, brain tumors, and bone tumors.

The Apo2L/TRAIL can be administered in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. Optionally, administration may be performed through mini-pump infusion using various commercially available devices.

It is contemplated that yet additional therapies may be employed in the methods. The one or more other therapies may include but are not limited to, administration of radiation therapy, cytokine(s), growth inhibitory agent(s), chemotherapeutic agent(s), cytotoxic agent(s), tyrosine kinase inhibitors, ras farnesyl transferase inhibitors, angiogenesis inhibitors, and cyclin-dependent kinase inhibitors which are known in the art and defined further with particularity in Section I above.

Preparation for chemotherapeutic agents may be used according to manufacturers' instructions or as determined empirically by the skilled practitioner. Preparation for such chemotherapy are also described in Chemotherapy Service Ed., M. C. Perry, Williams & Wilkins, Baltimore, Md. (1992).

In another embodiment of the invention, articles of manufacture containing materials useful for the treatment of cancer are provided. In one aspect, the article of manufacture comprises (a) a container comprising Apo2L/TRAIL (preferably the container comprises the Apo2L/TRAIL and a pharmaceutically acceptable carrier or diluent within the container); and (b) a package insert with instructions for treating cancer, wherein the instructions provide information such as that recited in the attached drawing sheets. Optionally, the package insert comprises information concerning administration, side effects, and/or advisory warnings, etc. set forth by the applicable regulatory agency, such as the FDA.

In all of these aspects, the package insert is on or associated with the container. Suitable containers include, for example, bottles, vials, syringes, etc. The containers may be formed from a variety of materials such as glass or plastic. The container holds or contains a composition that is effective for treating the cancer and may have a sterile access port (for example the container may be an intravenous solution bag or a vial having a stopper pierceable by a hypodermic injection needle). The article of manufacture may further comprise an additional container comprising a pharmaceutically acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

### EXAMPLES

Commercially available reagents referred to in the examples were used according to manufacturer's instructions unless otherwise indicated.

### Methods and Materials

Apo2L/TRAIL:

recombinant human Apo2L/TRAIL (“rhApo2L/TRAIL” or Dulanermin), consisting of amino acids 114-281 of Supplemental FIG. 11 (SEQ ID NO:1), was manufactured and formulated by Genentech, Inc., South San Francisco, Calif. as described in WO 01/00832 published Jan. 4, 2001 and WO 03/042344 published May 22, 2003. Recombinant soluble Flag-tagged human Apo2L/TRAIL was prepared according to a published method (Ashkenazi et al., J. Clin. Invest., 104:155-162 (1999); Kischkel et al., Immunity, 12:611-620 (2000)) (referred to in the examples below and in the figures as “Apo2L.M2”).

Mouse Models:

C57BL/6 (wildtype) mice were obtained from the Jackson Laboratory and C57BL/6.Rag2^{-/-} mice were obtained from Taconic, Inc. C57BL/6.DR5^{-/-} (Diehl, et al., Immunity, 21:877-889 (2004)) and C57BL/6.TNFR1^{-/-}TNFR2^{-/-} mice were bred and maintained at Genentech, Inc. under specific pathogen-free conditions. All animal experiments were reviewed and approved by the Institutional Animal Care and Use Committee at Genentech, Inc.

Fibrosarcoma Tumor Initiation:

C57BL/6 (wildtype) or C57BL/6.DR5^{-/-} mice were inoculated subcutaneously in the hind flank with 200 mg of methylcholanthrene (MCA) (Sigma-Aldrich) in 0.1 mL of corn oil, as previously described (Koebel, et al., Nature, 450:903-907 (2007)). Mice were assessed weekly for tumor development from 90 days after MCA treatment.

Cell Lines and Tumor Transplant Models:

Fibrosarcoma cell lines were created by mechanically dissociating primary tumor tissue in medium containing 2.5% heat inactivated FBS (fetal bovine serum) containing Liberase Blendzyme 2 (Roche Applied Biosystems). Single cell suspensions were obtained by pipetting the tissue pieces for 20 minutes at room temperature, as previously described (Koebel, et al., supra; Wilson, et al., Blood, 102:2187-2194 (2003)). EDTA (pH 7.2) was added for 5 minutes to disrupt cell clusters and to inhibit the enzymatic activity. Undigested fragments were removed by filtering. Cell pellets were resuspended in RPMI medium supplemented with L-glutamine and 10% fetal bovine serum (FBS) under conditions of 5% CO₂ at 37° C. Identical culture conditions were used to maintain Lewis Lung tumor cells (ATCC). Mice were injected subcutaneously with 5×10⁶ cancer cells. Tumors were measured in two dimensions using a caliper. Tumor volume was calculated using the formula: V=0.5a×b², where a and b are the long and the short diameters of the tumor, respectively. For anti-tumor efficacy studies, mice bearing ˜200 mm³ tumors were randomly assigned into groups and injected intraperitoneally with Apo2L and M2, according to the dosing regimen described. Tumor-bearing mice were sequentially administered intraperitoneally with 10 mg/kg of Apo2L followed by 10 mg/kg of the anti-Flag antibody (M2) (Sigma). Apo2L or M2 alone showed no anti-tumor effect (data not shown).

Cell Viability and Caspase-3 Assays:

Cell viability following Apo2L/TRAIL treatment was determined in vitro using the Cell-titer Glo cell viability assay (Promega). Caspase-3/7 or 8 activity was measured in vitro using the Caspase-Glo 3/7 or Caspase-Glo 8 assay (Promega), according to manufacturer's instructions. For in vitro viability of caspase assays, Apo2L and M2 were combined sequentially at a 1:1 molar ratio. Ex vivo caspase-3 processing in tumor cells was monitored by flow cytometry using the cleaved caspase-3-specific antibody (clone C92-605, BD Pharmingen). Caspase-3 activation is represented as a fold-increase relative to control treated mice.

Endothelial Cell DR5 Expression Analysis:

To generate a single cell suspension, Lewis lung tumors (<500=³) or kidneys from wildtype or DR5-deficient mice were dissected and mechanically dissociated into small fragments. Dissociated tissue was resuspended in medium containing 2.5% heat inactivated FBS containing Liberase Blendzyme 2 (Roche Applied Biosystems), according to the same protocol described to generate tumor cell lines. Cell pellets were resuspended in PBS containing 2.5% bovine serum albumin containing anti-Fc γreceptor (FcγR□□IIB/III (clone 2.4G2, BD Pharmingen), anti-FcγRIV (clone 39A.1, Genentech Inc.) to block FcγR binding non-specifically to the antibodies used to characterize endothelial cells: anti-CD45 (clone 104, BDPharmingen), anti-DR5 (clone MD5.1, eBiosciences) and anti-CD31 (clone 390, BD Pharmingen). Cell populations were then analyzed using a FACScan (Becton Dickinson) using 7AAD (BD Pharmingen) to exclude dead cells.

Immunohistochemistry:

Immunohistochemistry (IHC) was performed on 5 micron thick formalin-fixed paraffin embedded tissue sections mounted on glass slides. Slides for DR5 and panendothelial cell marker were deparaffinized in xylene and rehydrated through graded alcohols to distilled water. Slides were pretreated with Target Retrieval solution (Dako; Carpinteria, Calif.) for 20 minutes at 99° C. Slides were treated with KPL blocking solution (Kierkegaard and Perry Laboratories; Gaithersburg, Md.) and avidin/biotin block (Vector; Burlingame, Calif.) respectively. Nonspecific IgG binding was blocked with TBST containing 1% bovine serum albumin (Roche; Basel, Switzerland) and 10% normal goat serum, for DR5 IHC, or 10% normal rabbit serum for panendothelial cell marker IHC (Life Technologies; Carlsbad, Calif.). Primary antibodies were used at 10 μg/ml for DR5 (clone MD5-1, BD Biosciences; Franklin Lakes, N.J.) and 2 μg/ml for panendothelial cell marker (clone MECA-32, BD Biosciences, NJ). Slides were incubated in primary antibody for 60 minutes at room temperature. Slides were rinsed and incubated for 30 minutes with either biotinylated goat anti-hamster or biotinylated rabbit anti-rat secondary antibodies (Vector, CA) diluted to 7.5 μg/ml. Slides were then subsequently incubated in Vectastain ABC Elite reagent (Vector, CA) and Pierce metal enhanced DAB (Thermo Scientific; Worcester, Mass.), counterstained, dehydrated and coverslipped. Cleaved caspase 3 IHC (Asp175) was performed on the Ventana Discovery XT (Ventana Medical Systems; Tucson, Ariz.) autostainer utilizing cell conditioner 1, standard treatment. Primary antibody, cleaved caspase 3 (Asp175) (Cell Signaling Technologies; Danvers, Mass.) was used at a concentration of 0.06 μg/ml and incubated for 3 hours at 37° C. Ventana DABMap (Ventana Medical Systems; AZ) was used as the detection system.

Quantitation of Cleaved Caspase-3 Immunohistochemistry:

Images were acquired by the Olympus Nanozoomer automated slide scanning platform (Olympus America, Center Valley, Pa.) at 200× final magnification. Tumor-specific areas were analyzed in the Matlab software package (Mathworks, Natick, Mass.) as individual 24-bit RGB images. The brown DAB-specific staining was isolated from the Hematoxylin counterstain using a blue-normalization algorithm as described by Brey, et al., J. Histochem. Cytochem., 51:575-584 (2003)). Area measurements for both DAB and Hematoxylin positive areas were reported.

In Vivo Near Infrared Fluorescence Imaging:

Two hours after treatment with Apo2L/TRAIL or PBS mice (n=3 to 5/treatment group) were injected intravenously with the fluorescent blood pool marker AngioSense680IVM (PerkinElmer). The temporal distribution of AngioSense680IVM within tumors and neighboring tissue was measured by visualizing fluorescence (650 nm excitation/700 nm emission) with a Kodak 4000 FX Pro imaging system (CareStream Health) and quantifying fluorescence intensities within regions of interest placed over tumor or adjoining tissue normalized to time=0, (I_{ROTt=x}−I_{BG}) (I_{ROIt=0}−I_{BG}). At each indicated time point, animals were anesthetized under isoflurane with body temperature maintained at 37° C. and imaged.

### Experimental Results and Data

Murine cancer cells express DR5 but do not respond to Dulanermin, a trimeric recombinant soluble version of human Apo2L/TRAIL which has been evaluated in certain clinical trials (Ashkenazi et al., J. Clin. Invest., 104:155-162 (1999); Herbst et al., J. Clin. Oncol., 2010)) (Supplementary FIG. 1a, 1b, 1c). In the experiments conducted herein, it was observed that crosslinking of a Flag epitope-tagged version of Apo2L/TRAIL into oligomers with an anti-Flag antibody enabled proapoptotic signaling in a range of mouse cancer cell lines. These included Renca331 cells (Supplementary FIG. 1b), which are particularly sensitive to membrane-bound Apo2L/TRAIL (Seki et al., Cancer Res., 63:207-213 (2003)), as well as Lewis lung carcinoma (LLC) cells (Supplementary FIG. 1c).

To determine the efficacy of this cross-linked form of Apo-2 ligand in vivo, mouse LLC cells were implanted into C57BL/6 wildtype recipient mice and the animals were treated with a single dose of crosslinked Apo2L/TRAIL. Surprisingly, a striking hemorrhagic appearance was observed in tumors within 24 hours after treatment (FIG. 1a). Considering that LLC tumors are relatively resistant to anti-angiogenic therapy (Shojaei et al., Nat. Biotechnol., 25:911-920 (2007)), the effect of Apo2L/TRAIL suggested a more acute impact on the tumor vasculature. Histological examination confirmed extensive hemorrhage throughout the tumor, as well as widespread tumor cell death (FIG. 1b).

Immunohistochemical staining with the mouse endothelial-cell marker, Meca-32 (Hallmann et al., Dev. Dyn., 202:325-332 (1995)), revealed severe disruption of the tumor vasculature by Apo2L/TRAIL (FIG. 1c). To confirm these histological observations, a non-invasive near infrared fluorescence imaging technique that longitudinally monitors vascular integrity was utilized. Tumor-bearing wildtype and DR5-deficient mice were treated with Apo2L/TRAIL, injected intravenously with the blood pool probe AngioSense680IVM, then imaged over time. In wildtype, but not DR5-deficient, recipient mice, Apo2L/TRAIL induced rapid accumulation (within 3-6 hours) of the probe into LLC tumors, indicative of vascular disruption (FIG. 1d and Supplementary FIG. 2).

Remarkably, the effects of Apo2L/TRAIL on the tumor vasculature were completely abrogated upon implantation of the LLC tumor cells in DR5-deficient mice (FIG. 1a-d). Given this result, it appeared that the biological effect of Apo2L/TRAIL on the tumor-associated stromal compartment may be direct. Previous reports have suggested that Apo2L/TRAIL can induce apoptosis in endothelial cells. However, the majority of these studies were carried out using cultured endothelial cells, and arrived at conflicting conclusions about the effects of Apo2L/TRAIL in vitro (Li et al., J. Immunol., 171:1526-1533 (2003); Marini et al., BMC Cancer, 5:5 (2005); Chan et al., Circ. Res., 106:1061-1071 (2010); Chen et al., Biochem. Biophys. Res. Commun., 391:936-941 (2009)). One study reported disruption of tumor vasculature in mice injected with adenovirus-transduced human CD34+ cells engineered to express a membrane-bound form of Apo2L/TRAIL (Lavazza et al., Blood, 115:2231-2240 (2010)). However, it remained unclear whether this effect was the direct result of proapoptotic DR5 activation in endothelial cells, or an indirect consequence of targeting DR5 in the malignant tumor-cell compartment. Indeed, the introduction of these modified human cells into mice may also elicit responses in the tumor microenvironment that are not strictly attributable to proapoptotic DR5 signaling.

To further evaluate the relationship between the observed effects on the tumor vasculature and DR5 activation in tumor-associated endothelial cells (TECs), LLC tumors grown in wildtype or DR5-deficient recipients were dissociated and the isolated cells were stained for flow cytometric analysis with antibodies to three markers: DR5; the leukocyte common antigen, CD45; and the endothelial cell-associated antigen, CD31 (Tang et al., J. Biol. Chem., 268:22883-22894 (1993)). Differential CD45 and CD31 expression were used to broadly define tumor-associated leukocytes (CD45^{high}), an enriched tumor epithelial cell fraction (CD45^{low}CD31^{low}), and TECs (CD45^{low}CD31^{high}). DR5 protein expression was detected on CD45^{neg} epithelial cells from tumors grown in wildtype or DR5-deficient mice, but not on CD45^{high} leukocytes from tumors grown in either strain (Supplementary FIG. 3) (Tang et al., supra). Importantly, DR5 expression was also observed on CD45^{low}CD31^{high} TECs from tumors grown in wildtype but not DR5-deficient mice (FIG. 2a). By contrast, significant DR5 expression was not detected on CD45^{low}CD31^{high} endothelial cells isolated from normal mouse kidney (FIG. 2b). Immunohistochemistry confirmed DR5 expression on endothelial cells within the tumor stroma of wildtype, but not DR5-deficient, mice (FIG. 2c). Of note, malignant epithelial cells expressed DR5 regardless of DR5 status in the stromal compartment.

Endothelial cells are phenotypically and functionally diverse, with differential tissue-specific surface marker expression and gap-junction properties (Dejana et al., Nat. Rev. Mol. Cell Biol., 5:261-270 (2004); Pober et al., Nat. Rev. Immunol., 7:803-815 (2007)). Consistent with the lack of DR5 expression by endothelial cells in normal tissues, there was not any evidence of vascular disruption or hemorrhage outside of the tumor microenvironment in Apo2L/TRAIL-treated mice. The apparent specificity of DR5 expression by TECs as compared to normal endothelial cells may reflect environmental conditions within the tumor such as hypoxia—a condition that has been shown to modulate DR5 expression in cancer cells (Mahajan et al., Carcinogenesis, 29:1734-1741 (2008)).

To assess proapoptotic signaling in TECs, mice harboring LLC tumors were treated with the Apo2L/TRAIL and monitored for the appearance of apoptotic markers in the tumor endothelium. Serial sections of tumor tissue were stained with Meca-32 to localize TECs, or with an antibody specific to active (cleaved) caspase-3 as a marker of proapoptotic signaling. Rapid generation of active caspase-3 was detected in TECs within two hours after Apo2L/TRAIL treatment (FIG. 2d). Some areas of active caspase-3 staining appeared in tumor epithelial cells regardless of treatment, suggesting spontaneous focal apoptosis—a common occurrence in mouse tumors. By 24 hours after Apo2L/TRAIL treatment, extensive active caspase-3 staining could be seen throughout the tumor (FIG. 2e; Supplementary FIG. 4). At early time points, little caspase-3 activity was present overall within tumor epithelial cells (FIGS. 2d and e), suggesting that Apo2L/TRAIL-induced apoptosis in TECs preceeded, and was independent of, apoptosis in the malignant cell compartment. Apo2L/TRAIL did not induce TEC apoptosis in LLC tumors grown in DR5-deficient mice (Supplementary FIG. 4), confirming DR5-dependent signaling in TECs.

In addition to proapoptotic signaling, engagement of death receptors under certain circumstances can activate non-apoptotic pathways such as the nuclear factor kB (NF-kB) cascade, which can promote cytokine and chemokine production among other cellular effects (Wilson et al., Nat. Immunol., 10:348-355 (2009)). Tumor necrosis factor alpha (TNFα), which often is produced in response to NF-kB activation, has been reported to trigger dramatic tumor vascular effects (Corti et al., Ann. NY Acad. Sci., 1028:104-112 (2004); ten Hagen et al., Immunol. Rev. 222:299-315 (2008)). To examine whether the impact of DR5 activation on the tumor vasculature might be exerted indirectly, for example via TNFa, TNF receptor (TNFR) 1 and 2 double-deficient mice were implanted with LLC tumors and treated with Apo2L/TRAIL. The appearance and incidence of tumor vascular disruption induced by Apo2L/TRAIL in TNFR1/2-deficient mice were indistinguishable from those in wildtype mice, and absent in DR5-deficient recipients (FIGS. 2f and g). In accordance, TNFR1/2 deficiency in the stromal compartment had no effect on Apo2L/TRAIL-induced caspase-3 activation in tumor epithelial cells (Supplementary FIG. 5).

Methylcholanthrene (MCA)-induced fibrosarcomas were generated in wildtype and DR5-deficient mice and cell lines from the tumors were established. The DR5-expression status of these tumor cell lines was confirmed by flow cytometry (FIG. 3a). Wildtype or DR5-deficient MCA tumors were then grown by implanting these tumor cell lines in DR5-positive or DR5-negative recipient mice. Treatment with Apo2L/TRAIL induced significant tumor hemorrhage by 24 hours independently of DR5 expression in malignant cells (FIG. 3b); in contrast, this phenotype was completely absent in tumors with DR5-deficient stroma. Meca-32 and activated caspase-3 staining confirmed proapoptotic signaling in TECs within tumors expressing or lacking DR5 in the malignant cell compartment (FIGS. 3c and 3d). These data demonstrate that disruption of the tumor vasculature by Apo2L/TRAIL occurs independently of DR5 activation in malignant cells. Moreover, Apo2L/TRAIL treatment increased caspase-3 activity in both wildtype and DR5-deficient tumor cells, perhaps reflecting secondary, DR5-independent apoptosis caused by a substantial disruption of the tumor vasculature.

The anti-cancer efficacy of Apo2L/TRAIL in mice bearing wildtype or DR5-deficient tumors was further evaluated. In vitro assays for activation of caspase-8, caspase-3/7, or loss of cell viability confirmed the lack of proapoptotic signaling in DR5-deficient MCA tumor cells treated with Apo2L/TRAIL (FIG. 4a). However, when implanted in DR5-positive mice, DR5-deficient fibrosarcomas showed significant caspase-3 activation in response to Apo2L/TRAIL (FIG. 4b). Moreover, Apo2L/TRAIL treatment significantly delayed tumor growth in mice transplanted with either wildtype or DR5-deficient fibrosarcomas (FIGS. 4c and d). In both cases, extensive, hemorrhagic tumor necrosis following Apo2L/TRAIL treatment was noted (Supplementary FIG. 6), suggesting that death of malignant cells occurred as an indirect consequence of tumor vascular disruption. These data demonstrate that DR5 activation in TECs contributes to anti-tumor efficacy in a manner that is distinct and separable from DR5-dependent tumor-cell apoptosis. Of note, Apo2L/TRAIL did not induce significant propaoptotic signaling in cancer cells upon implantation of wildtype fibrosarcomas in DR5-deficient mice (Supplementary FIG. 7). Similar results were seen in the Lewis lung carcinoma model. Tumor initiation and growth in the absence of treatment were not affected by the DR5 status of the recipient mice (Supplementary FIG. 8); however, as observed in the fibrosarcoma model, the anti-tumor effect of Apo2L/TRAIL was contingent on DR5 expression in stromal TECs. Therefore, in the fibrosarcoma and lung carcinoma models used in this study, DR5 activation on TECs is likely to be the primary mechanism for tumor inhibition by Apo2L/TRAIL. Similar tumor vascular disruption by Apo2L/TRAIL in a human lung cancer xenograft model, as well as a genetic mouse model of human pancreatic cancer was also observed (Supplementary FIGS. 9 and 10).

What is claimed is:

1. A method of disrupting tumor associated vasculature in mammalian tissue or cells, comprising exposing said tissue or cells to a therapeutically effective amount of Apo2L/TRAIL polypeptide or death receptor agonist antibody.

2. The method of claim 1 wherein endothelial cells comprising the tumor associated vasculature express DR5 receptor.

3. The method of claim 1 wherein the mammalian tissue or cells comprise tumor or cancer cells that do not express DR5 receptor.

4. The method of claim 1 wherein the mammalian tissue or cells comprise tumor or cancer cells that express DR5 receptor and are resistant to apoptosis induction by said DR5 receptor.

5. The method of claim 1 wherein said Apo2L/TRAIL polypeptide is an oligomer or cross-linked form of Apo2L/TRAIL.

6. The method of claim 1 wherein said death receptor agonist antibody is an anti-DR5 monoclonal antibody.

7. A method of treating cancer in a mammal, comprising administering to said mammal a therapeutically effective amount of Apo2L/TRAIL polypeptide or death receptor agonist antibody to disrupt tumor associated vasculature in the mammal.

8. The method of claim 7 wherein said Apo2L/TRAIL polypeptide or death receptor agonist antibody disrupts said vasculature and inhibits blood flow to the tumor.

9. The method of claim 7 wherein endothelial cells comprising the tumor associated vasculature express DR5 receptor.

10. The method of claim 7 wherein the mammal's tumor or cancer cells do not express DR5 receptor.

11. The method of claim 7 wherein the mammal's tumor or cancer cells express DR5 receptor and are resistant to apoptosis induction by said DR5 receptor.

12. The method of claim 7 wherein one or more chemotherapeutic agents or radiation therapy is further administered to said mammal.

13. The method of claim 7 wherein anti-VEGF antibody is further administered to said mammal.

14. The method of claim 13 wherein said anti-VEGF antibody is bevacizumab.

15. The method of claim 7 wherein said Apo2L/TRAIL polypeptide is an oligomer or cross-linked form of Apo2L/TRAIL.

16. The method of claim 7 wherein said death receptor agonist antibody is an anti-DR5 monoclonal antibody.

17. The method of claim 7 wherein said cancer is lung carcinoma or pancreatic cancer.

18. Use of Apo2L/TRAIL polypeptide or death receptor agonist antibody in the manufacture of a medicament for disrupting tumor associated vasculature or for the treatment of cancer.

19. The use of claim 18 wherein said Apo2L/TRAIL polypeptide is an oligomer or cross-linked form of Apo2L/TRAIL.

20. The use of claim 18 wherein said death receptor agonist antibody is an anti-DR5 monoclonal antibody.

21. The use of Apo2L/TRAIL polypeptide or death receptor agonist antibody in the manufacture of a kit for use in treating cancer.

22. A kit for use in the treatment of cancer, comprising (a) a container comprising Apo2L/TRAIL polypeptide or death receptor agonist antibody and a pharmaceutically acceptable carrier or diluent within the container; and (b) a package insert with instructions for administering said Apo2L/TRAIL polypeptide or death receptor agonist antibody to disrupt tumor associated vasculature in a human patient having cancer.
